(19) United States
(12) Patent Application Publication
(10) Pub. No.: US 2019/0026425 A1
(43) Pub. Date: Jan. 24, 2019

(54) CURATED GENETIC DATABASE FOR IN SILICO TESTING, LICENSING AND PAYMENT

(71) Applicant: YouGene, Inc., New York, NY (US)

(72) Inventors: Ryan Downs, Alexandria, VA (US); Siddharth Murali, Dayton, NJ (US); Roger C. Hahn, Fairfax, VA (US)

(73) Assignee: YouGene, Inc., New York, NY (US)

(21) Appl. No.: 16/131,518

(22) Filed: Sep. 14, 2018

Related U.S. Application Data
(63) Continuation-in-part of application No. 15/390,306, filed on Dec. 23, 2016.
(60) Provisional application No. 62/387,364, filed on Dec. 24, 2015.
(60) Provisional application No. 62/278,891, filed on Jan. 14, 2016.

(51) Int. Cl. G06F 19/18; G06Q 20/06; G06F 19/24; G06F 19/22; G06F 19/28; G16H 10/40; G16H 15/00; G16H 50/20; G16H 50/30; G16H 50/70; H04L 9/06; G06F 17/30

(52) U.S. Cl. CPC G06F 19/18; G06Q 20/06; G06F 19/24; G06F 19/22; G06F 19/28; G16H 10/40; H04L 2209/38; G16H 50/20; G16H 50/30; G16H 50/70; H04L 9/0637; G06F 17/3056; G06F 17/30194; G16H 15/00

(57) ABSTRACT

This disclosure relates to methods and systems for a curated genetic variant database and systems and methods for submitting new genetic tests based on the information in the curated database. The methods and systems of the invention further provide for a single curated variant database that allows curation of genetic variants while protecting the proprietary nature of the information submitted to the database. The system and methods also provide for submission of new genetic tests based on genetic variants, conducting genetic tests, and for determining payments to submitters and test developers based on the genetic tests.

### FIELD OF THE INVENTION

This disclosure relates to methods and systems for a curated genetic variant database and systems and methods for submitting new genetic tests based on the information in the curated database. The methods and systems of the invention further provide for a single curated variant database that allows curation of genetic variants while protecting the proprietary nature of the information submitted to the database. The system and methods also provide for submission of new genetic tests based on genetic variants, conducting genetic tests, and for determining payments to submitters and test developers based on the genetic tests.

### BACKGROUND

Rapid adoption of NGS-based tests in both research and clinical practice is leading to identification of an increasing number of genetic variants. Understanding the clinical significance of these genetic variants and making such information available in a medical-grade database is critical to enabling for making that knowledge available for widespread clinical use. Limited access to highly-annotated and transparently-sourced content has hindered laboratories in their efforts to leverage next generation sequencing. Clinical laboratories require access to high-quality content, preferably clinically validated and evidence based information that is not widely available today. The data that is available through various public and commercial databases is often filled with few parameters to ensure quality. Additionally, a 2013 report published by the American College of Medical Genetics and Genomics, warns that few, if any, of the databases are curated to a level necessary for clinical use. The FDA has pointed out that several genetic tests are inaccurate, leading to both false negatives and false positives, resulting in danger to patients and increased cost.

Crowdsourcing can leverage the resources of individuals to produce a centralized, open-sourced platform for the diverse community to share findings while a blockchain can provide an immutable shared ledger that tracks contributions to incentivize sustained and durable participation using tokens. The clinical significance of the genetic variants can be obtained by data curation spread across multiple stakeholders. Curation of the literature to produce a high-quality set of pathogenic variants is not trivial and one group could not independently keep pace with the ever expanding cancer genomics literature. Moreover, in the absence of appropriate incentives to encourage community data curation, different groups will be unwilling to participate in a shared community. Specifically, they will not aggregate, curate, interpret, findings.

Hence, there is a need for a network based on a private, permissioned blockchain to incentivize contributions to a shared database. There is further a need for systems that can ensure transparency in providing payments to parties that participate in the database according to their contributions.

There is a further need for a system that encourages submission of data to a single, curated, variant database, ensuring the accuracy of the data, while protecting the proprietary nature of the data by allowing the original submitters of information to receive a monetary benefit from the data submitted and by keeping proprietary information private as opposed to public. There is further a need for a system that encourages researchers to make their research publicly available, allowing additional parties to use the information in developing novel genetic tests, while ensuring that the researchers themselves are justly compensated for the work put into the underlying data. There is a need for a system that allows clinicians and insurers to have access to a comprehensive database of validated biomarkers for reimbursement, while allowing test developers to mitigate regulatory risk by using a validated database, inducing clinicians and insurers to participate while giving variant submitters an opportunity to reduce their IT footprint, free up cash, and enable more focus on finding new discoveries and products. There is also a need for a system that allows a patient to undergo a single genome sequencing, and then to use the single sequenced genome for multiple genetic tests, both using proprietary and non-proprietary data.

### SUMMARY OF THE INVENTION

A system and methods are provided for maintaining a curated database of genetic variants. In any embodiment, the system can include a genetic information database; wherein the genetic information database contains genetic variants and estimated effects of the genetic variants; and a curation application, wherein the curation application is connected to the genetic information database, and wherein the curation application is accessible by one or more curators and allows access to the genetic information database; wherein the curation application allows the one or more curators to curate the information in the genetic information database and allows the one or more curators to provide a curation score for information in the genetic information database.

In any embodiment, the information in the genetic information database is submitted by one or more submitting parties, and the one or more curators are not provided the identity of the submitting parties.

In any embodiment, there are at least two curators.

In any embodiment, the curation application provides each curator with ratings provided by each other curator.

In any embodiment, the system can provide a variant curation score as an average of curation scores provided by each curator.

In any embodiment a system can include a genetic information database; wherein the genetic information database contains genetic variants and estimated effects of the genetic variants; and a submission application; wherein the submission application is connected to the genetic information database; and wherein the submission application is accessible by one or more submitters; wherein the submission application allows the one or more submitters to submit the genetic variants and estimated effects of the genetic variants to the genetic information database; and wherein the submission application allows information to be submitted as a visible submission or an invisible submission; wherein the visible submission is accessible by any user and wherein the invisible submission is not accessible by any other submitter.

In any embodiment, the system further includes a curation application; wherein the curation application is connected to the genetic information database, and wherein the curation application is accessible by one or more curators and allows access to the genetic information database; wherein the curation application allows the one or more curators to curate the information in the genetic information database and allows the one or more curators to provide a curation score for information in the genetic information database; and wherein the curation application allows access for the one or more curators to both visible and invisible submissions.

In any embodiment, the system can include a curation request application; wherein the curation request application is in communication with the curation application; wherein the curation request application is accessible by one or more requesters; wherein the curation request application receives a curation request from the one or more requesters and transmits the curation request to the curation application; and wherein the curation request application receives a curation report from the curation application and transmits the curation report to the requester.

In any embodiment, the system includes a test developer application; wherein the test developer application is connected to the genetic information database; wherein the test developer application is accessible to one or more test developers; and wherein the test developer application allows the one or more test developers to access the information in the genetic information database that is submitted as a visible submission.

In any embodiment, the test developer application determines whether a test developer is a submitter of an invisible submission.

In any embodiment, the test developer application allows the test developer to access an invisible submission only if the test developer is the submitter of the invisible submission.

In any embodiment, the genetic information database is configured to calculate a variant score for information in the genetic information database.

In any embodiment, the variant score is calculated at least in part based on one or more of the group of: a number of submitters that have submitted the variant, a time factor, a data quality score, a curation score, a participation score, a credibility score, and whether the variant is a visible submission.

In any embodiment, the variant score is 0 for any invisible submission.

In any embodiment, the variant score can be calculated by an algorithm V=0*(Q+C+P+R); wherein V represents the variant score, O represents the time factor; Q represents the data quality score, C represents the credibility score, P represents the participation score, and R represents the curation score.

In any embodiment, the time factor O can be calculated by an algorithm O=1/F; wherein F is an order of submission by the submitter of the variant.

In any embodiment, the variant score can be calculated by an algorithm V=O*Q*R; wherein V represents the variant score, O represents a time factor; Q represents the data quality score, and R represents the curation score.

In any embodiment, a system can include a genetic information database; wherein the genetic information database contains genetic variants and estimated effects of the genetic variants; a test developer application, wherein the test developer application is connected to the genetic information database wherein the test developer application is accessible to one or more test developers; and wherein the test developer application allows the one or more test developers to access the information in the genetic information database; and a test submission application; wherein the test submission application is connected to a genetic data interpretations server; and wherein the test submission application allows the one or more test developers to submit a genetic test; wherein the genetic test includes instructions for determining the presence, absence, or likelihood of a genetic condition; and wherein the genetic test is based on one or more variants in the genetic information database.

In any embodiment, the genetic data interpretations server is connected to a remote application; wherein the remote application is connected to a genetic data storage server; wherein the genetic data storage server contains genetic data from one or more patients; and wherein the remote application is configured to carry out the instructions of the genetic test to determine the presence, absence or likelihood of the genetic condition for the patient.

In any embodiment, the system includes a payment application, wherein the payment application is configured to account for a payment from a payer party for conducting a genetic test using the genetic test submitted by the test developer, and to account for a payment to the test developer and a submitter for conducting the genetic test, wherein the submitter has submitted a variant to the genetic information database on which the genetic test has been based.

In any embodiment, the variants in the genetic information database are submitted by a submitter through a submission application; wherein the submission application allows the submitter to submit information as a visible submission or an invisible submission; and wherein the test developer application is configured to determine determines whether a test developer is a submitter of an invisible submission; and wherein the test developer application does not allow access for a test developer to the invisible submission unless the test developer application determines that the test developer is the submitter of the invisible submission.

In any embodiment, the payment to the test developer and the payment to the submitters are based, at least in part, on a variant score for each variant on which the genetic test is based.

In any embodiment, the variant score is based, at least in part, on one or more of the group of: a number of submitters that have submitted the variant, a data quality score, a curation score, a participation score, and whether the variant is a visible submission.

In any embodiment, the variant score is determined to be 0 if the variant is an invisible submission.

In any embodiment, the variant score can be calculated by an algorithm V=O* (Q+C+P+R); wherein V represents the variant score, O represents the time factor; Q represents the data quality score, C represents the credibility score, P represents the participation score, and R represents the curation score.

In any embodiment, a total variant score for a particular variant used in a genetic test can be given by an algorithm T=Σₖ₌₁ⁿVₖ; wherein T is the total variant score for the variant, Vₖ is the variant score for each submitter k that submitted the variant; and n is the total number of submitters that submitted the variant.

In any embodiment, the system can calculate a total variant points using an algorithm A=Σₖ₌₁ⁿTₖ; wherein Tₖ is the total variant score for a given variant k, and wherein m is the total number of variants used in the genetic test.

In any embodiment, the payment to the test developer for use of the genetic test can be calculated by an algorithm

$$D = \frac{U * M}{A + L} * L ;$$

wherein U is a price of the genetic test, M is a system value factor, A is the total variant points, and L is a test developer value.

In any embodiment, the system includes a curation application; wherein the curation application is connected to the genetic information database, and wherein the curation application is accessible by one or more curators and allows access to the genetic information database; wherein the curation application allows the one or more curators to curate the information in the genetic information database and allows the one or more curators to provide a rating for variants in the genetic information database; wherein the curation application allows access for the one or more curators to both visible and invisible submissions; and wherein the curation score is based on the rating provided for the variant by the one or more curators.

In a third embodiment, a system can comprise a genetic information database; wherein the genetic information database contains genetic variants and estimated effects of the genetic variants; a curation application, wherein the curation application is connected to the genetic information database, and wherein the curation application is accessible by one or more curators and allows access to the genetic information database; wherein the curation application allows the one or more curators to curate information in the genetic information database and allows the one or more curators to provide a curation score for the information in the genetic information database; a submission application; wherein the submission application is connected to the genetic information database; and wherein the submission application is accessible by one or more submitters; wherein the submission application allows the one or more submitters to submit the genetic variants and estimated effects of the genetic variants to the genetic information database; and a payment, application, wherein the payment application is programmed to account for a payment to the one or more curators and the one or more submitters.

In any embodiment, the payment application can account for the payment to the one or more curators each time a variant curated by a curator is viewed, each time a curator curates a variant, or a combination thereof.

In any embodiment, the payment application can account for the payment to the one or more submitters either each time a variant submitted by a submitter is used in a genetic test, each time a variant submitted by a submitter is viewed, or a combination thereof.

In any embodiment, the system can be programmed to use a block chain to account for payment to the one or more curators.

In any embodiment, the system can be programmed to update the blockchain each time a variant is curated by a different curator.

In any embodiment, the system can be programmed to account for the payment to the one or more curators by a fiat currency, a utility token, or a security token.

In any embodiment, a payment to the one or more submitters can be based on a variant score for a submission.

In any embodiment, the system can be programmed to use a block chain to account for the payment to the one or more submitters.

In any embodiment, the system can be programmed to account for the payment to the one or more submitters a fiat currency, a utility token, or a security token.

In any embodiment, the system can comprise a test submission application; wherein the test submission application is connected to a genetic data interpretations server; and wherein the test submission application allows one or more test developers to submit a genetic test; wherein the genetic test comprises instructions for determining a presence, absence, or likelihood of a genetic condition; and wherein the genetic test is based on one or more variants in the genetic information database.

In any embodiment, the genetic data interpretations server can be collocated with a genetic data storage server and a remote client; the genetic data storage server containing a genome or portion of a genome for one or more patients; the remote client programmed to conduct a genetic test based on information in the genetic data interpretations server.

In any embodiment, the payment application can be programmed to account for a payment to the one or more test developers each time a genetic test developed by the one or more test developers is conducted.

In any embodiment, the payment application can be programmed to account for a payment to the one more submitters each time a genetic test is conducted using a variant submitted by the one or more submitters, and to account for a payment to the one more curators each time a genetic test is conducted using a variant curated by the one or more curators.

In any embodiment, the system can he programmed to use a block chain to account for the payment to the one or more submitters.

In any embodiment, the system can be programmed to account for the payment to the one or more submitters by a fiat currency, a utility token, or a security token.

In any embodiment, the payment to the one or more submitters can be based on a price for the genetic test and a variant score for each variant submitted by the one or more submitters used in the genetic test.

In any embodiment, the payment application can be programmed to account for a payment from a payer party to the one or more test developers each time a genetic test developed by the one or more test developers is conducted, to the one or more curators each time a genetic test using a variant curated by the one or more curators is conducted, and to the one or more submitters each time a genetic test using a variant submitted by the one or more submitters is conducted.

In any embodiment, the payment application can account for a payment from a subscriber for viewing one or more variants in the genetic information database.

In any embodiment, the payment application can be programmed to distribute the payment from the subscriber to the one or more submitters and one or more curators according to an algorithm.

In any embodiment, the system can comprise a curation database, the curation database containing curation information submitted by the one or more curators.

### BRIEF DESCRIPTION OF THE DRAWINGS

FIG. 1 shows a schematic of a system for accounting for payments to contributors for conducting genetic tests.

FIG. 2 shows a schematic of a system for accounting for payments to contributors for conducting genetic tests using a cryptocurrency.

FIG. 3A-C illustrate example blockchain used to track genetic test usage and payments.

FIG. 4 is an example of a dynamically evolving blockchain for distributing royalty payments to contributors.

FIG. 5 shows a system for managing a genetic test development and genetic test conducting system.

FIG. 6 shows an overview of a system for managing a curated genetic variant database.

FIG. 7 shows a schematic of a system for submission of data to a curated genetic variant database and for curation of the data submitted.

FIG. 8 shows an example of curation of genetic information.

FIG. 9 shows a schematic of a system for the use of data from a curated database in developing and submitting a genetic test.

FIG. 10 shows one embodiment of a system for carrying out a genetic test.

FIG. 11 shows a schematic of a system for accounting for payments to test developers and data submitters based on usage of a genetic test.

FIG. 12 illustrates curation of the information within the system by separate expert curating groups.

FIG. 13 shows a schematic of a system for curation of genetic variants and use of the genetic variants in a genetic test.

FIG. 14 is a pyramid illustrating the level of evidence needed for FDA approval.

### DETAILED DESCRIPTION

### Definitions

Unless defined otherwise, all technical and scientific terms used herein generally have the same meaning as commonly understood by one of ordinary skill in the relevant art.

The articles “a” and “an” are used herein to refer to one or to more than one (i.e., to at least one) of the grammatical object of the article. By way of example, “an element” means one element or more than one element.

The term “absence” of a genetic condition refers to a patient that does not have, and will not develop, a particular condition.

The term “access” or “accessible” refers to the ability of a party to obtain information from one or more servers, databases, applications or other electronic media. The access may allow the party to view all or only some of the data provided on the server, database, application or media.

The term “account for a payment” refers to the creation of a record detailing the obligation of one user of the systems or methods described herein to pay another user of the systems or methods described here. The actual receipt of financial funds is not necessary to complete a “payment.” Rather, the financial funds can be escrowed by an administrator or another party who receives funds from one user and holds them for benefit of another user. Alternatively, payment can be completed by updating a log, database, or sending a notification that payment is due from one party to another where the transfer of financial funds can occur at some later time. However, a “payment” can also occur by the transfer of financial funds from one user to another user.

The term “administrator” or “administrator user” refers to one or more individuals or parties responsible for maintaining the soundness and usability of the systems and methods described herein.

The term “biomarker” refers to a substance that whose quantitative or qualitative characteristics are used to determine a biological state or the presence or risk for a disease or condition. Biomarkers expressly include genomic information as indicated by a sequence or presence of certain nucleotide bases in a DNA molecule. Other express and non-limiting examples of biomarkers include quantitative or qualitative information regarding single nucleotide polymorphisms (SNPs), whole genome sequencing, genetic mutations, genetic linkage disequilibrium, metabolite information, proteomic information and lipidomic information.

A “blockchain” is a system that enables every participant to possess their own replicated copy of a distributed ledger. The distributed ledger contains transactions and ownership information. In addition to ledger information being shared, the processes which update the ledger are also shared.

The term “cloud” refers to any network or server that exists as a separate entity from the internet.

The term “collocated” refers to two or more servers, databases, computers, software applications, or any other computing module being in the same location. The same location can mean on the same server, virtual instance, or computer, on a single intranet, or located in the cloud behind the same firewall. “Collocated” can also refer to two or more modules configured such that data can be transmitted between the two or more modules without transmitting the data over the internet. “Collocated” can also refer to two or more modules configured such that one of the modules is embedded within the other module.

The term “comprising” includes, but is not limited to, whatever follows the word “comprising.” Thus, use of the term indicates that the listed elements are required or mandatory but that other elements are optional and may or may not be present.

To “conduct” a genetic test refers to scanning a genome of a patient and providing results to the patient or clinician.

The term “consisting of” includes and is limited to whatever follows the phrase “consisting of.” Thus, the phrase indicates that the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

The term “control server,” “control application,” or “CS” refers to a server or application configured to communicate with other servers, databases, or applications and to send and receive information from the other servers, databases or applications.

The term “curation,” “curated,” “curate,” or “curator” refers to the process of review of a genetic variant and an estimated result of a genetic variant by a qualified expert based on data submitted to a system.

A “curation application” refers to any application, server, or other interface that allows a curator to review data submitted concerning a particular genetic variant and the estimated result of the genetic variant. The curation application can also allow the curator to provide a level of confidence the curator has in the estimated result of the genetic variant.

A “curation database” is a database containing information submitted by one or more curators concerning the effects of one or more genetic variants.

A “curation request application” refers to any application, server, or other interface that allows a request to request curation of submitted data.

A “curation score” refers to a numerical value assigned to a genetic variant and the estimated result of the genetic variant based on the level of confidence of one or more curators in the estimated result.

A “dapp” is a decentralized application that runs on a blockchain platform such as Ethereum, Qtum, NEO, or HyperLedger Fabric. The platforms are the foundation of the blockchain, providing the technology, protocols and a computer network.

The term “database” refers to any organization of data or information that can be queried.

A “data quality score” is a quantitative value based on an objective level of confidence in information submitted to a genetic information database.

A “distributed ledger” is a consensus of replicated, shared, and synchronized digital data geographically spread across multiple computers, sites, countries, or institutions.

The term “estimated effect of a variant” refers to a phenotypic result that a party believes to be of a particular genetic variant. An “estimated effect of a variant” can refer to a phenotype that a submitter of the variant believes will result from a genetic variant, whether or not curators or other parties agree with the estimate.

A “fiat currency” is a currency that is issued and backed by a government, and is not backed by a commodity.

A “genetic data interpretations server” or “GDIS” is a server or database containing instructions on interpreting genetic or other biological data.

A “genetic data storage server” or “GDSS” is a server or database containing genetic or other biological data pertaining to one or more patients.

A “genetic information database” is a server or database containing genetic variants and estimated results of the genetic variants submitted by one or more parties.

A “genetic test” is a diagnostic test that provides results based on a genome or portion of a genome of a patient.

“Genetic usage information” refers to the information necessary for conducting a genetic test. As use herein, genetic usage information can refer to a prescription for a genetic test, the biomarkers to be searched during a genetic test, and/or the portions of the genome to be scanned during a genetic test.

A “genetic variant” or “variant” is a particular portion of a genetic code, wherein some percentage of the population will have a different sequence of base pairs at that portion than others. Structural variants can refer, without limitation, to insertions of base pairs into the genetic code, deletions of base pairs from the genetic code, rearrangements of base pairs within the genetic code, duplications of portions of the genetic code, translocations of one or more base pairs, inversions of portions of the genetic code, or mutations of one or more base pairs within the portion of the genetic code.

The term “information” refers to any algorithm, script, association, or any other data that can be stored by a computer.

An “invisible submission” or “invisible information” refers to information provided in a database that is not accessible by certain users of the database, and may only be accessed by authorized or specified users.

The term “likelihood” of a genetic condition refers to a probability that a specific patient will develop a condition in their lifetime.

The term “patient” or “patient user” refers to an individual, human or animal, from whom diagnostic information concerning biomarkers is taken.

The term “patient identification information” refers to any data that contributes to the personal identity of an individual.

A “participation score” refers to a quantitative value based on the number of variants a particular party has submitted to a genetic information database.

The term “payer party” or “payer party user” refers to an insurer or other party that is responsible for at least a partial payment to another user of the system and methods described herein. The payer party in addition to an insurance company can include a patient receiving the benefit of a diagnostic service. In any embodiment, the payer party can also refer to a patient if the patient is responsible for making a particular payment.

A “payment application” is any application, server, or other interface that can account for payments to and from any users of the system.

The term “phenotypic information” refers to any manifestation of a particular genotype.

A “prescription for a test” is a request by any party to search or analyze biological information.

The term “presence” of a genetic condition refers to a patient actually having a condition or a patient that will eventually develop the condition.

The term “price of a genetic test” refers to an amount of money paid by a payer party for conducting a genetic test.

The term “programmed,” when referring to a processor, can mean a series of instructions that cause a processor to perform certain steps. For example, a processor can be “programmed” to set functions, parameters, variables, or instructions.

The term “record” refers to a set of data present in a database that is associated with the same object such as a patient or biomarker.

A “remote client,” “remote client application,” “remote application,” or “RCA” is an application collocated with a genetic data storage server, and configured to receive instructions for interpreting genetic data and to interpret the genetic data according to the instructions.

A “requester,” as used herein, refers to a party requesting a system to provide curation of data submitted.

A “security token” is a token that represents ownership of an asset, such as debt or company stock.

The term “server” means any structure capable of storing digital information. As used herein, “server” can also refer to a database, application, intranet, virtual instance, or other digital structure.

A “submission application” refers to any application, server, or other interface that allows a submitting party to provide information to a genetic information database.

The terms “submit” or “submission” refer to the process of providing information to a system.

A “submitting party” or a “submitter” is a party that submits data to a genetic information database.

A “subscriber” is a party that pays for access to information in a system.

A “test developer” is a party that uses information in a genetic information database for the purposes of creating a genetic test and/or a party that creates a genetic test.

A “test developer application” refers to any application, server, or other interface that allows a test developer or other user to access information in a genetic information database, and/or to submit electronic instructions for carrying out a genetic test on a patient's genetic information. In any embodiment, separate applications can be used for accessing the information in the genetic information database and for submitting electronic instructions for carrying out the genetic test. In such a case, the application that allows a test developer to access the information in the genetic information database is the “test developer application” and the application that allows submission of the genetic test is a “test submission application.” In any embodiment, a single application can operate all of the functions of the “test developer application.”

A “test submission application” refers to any application, server, or other interface that allows a test developer to submit electronic instructions that, when carried out, conduct a genetic test a genetic data in the system.

A “third party request application” is an application collocated with a genetic data storage server and remote client that allows a request for a test to be made directly to the remote client.

The term “user” refers to any party or agent of a party who sends or receives information from the systems described herein or by means of the methods described herein.

A “utility token” is a digital token which is not sold by an issuing company for value or do not involve an investment of money. A utility token has a specific function that is only available to token holders. Utility tokens do not entitle the holder to a share of profits and/or losses, or assets and/or liabilities.

A “variant score” is a calculated number attributable to submission of a particular genetic variant.

The term “to view” information refers to accessing the information in a readable format.

A “visible submission” or “visible information” refers to information provided in a database that is accessible to any user of the database.

### Curated Database

The systems and methods described herein provide for the development of new genetic tests and genetic variant curation data based on genetic variants submitted to a curated database. The systems and methods also allow for curation of variant information while protecting the proprietary nature of information submitted to the system.

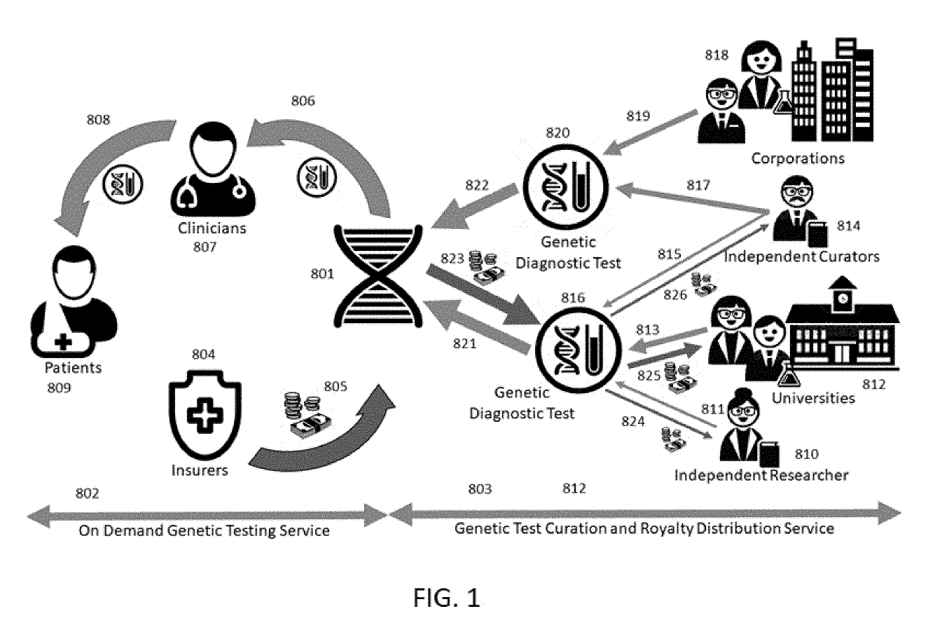

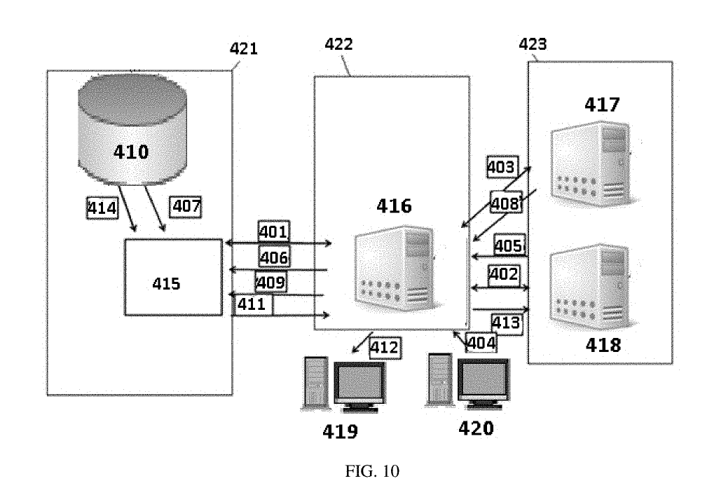

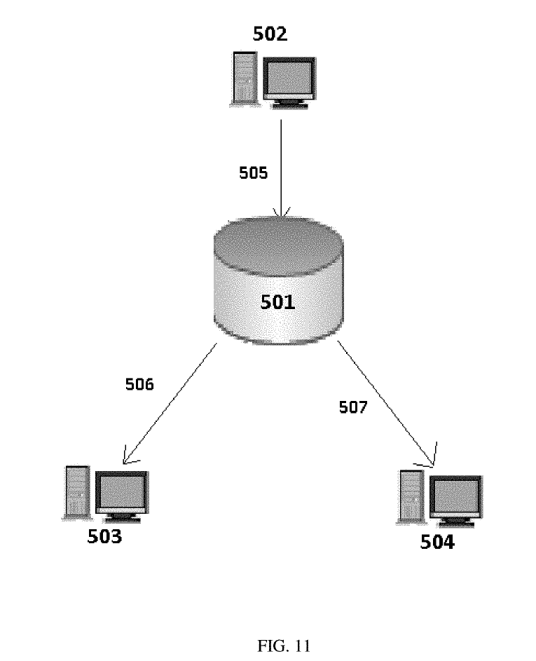

FIG. 1 illustrates a non-limiting embodiment of a system for curation of genetic variants, genetic testing, and distribution of royalties to each party. The genetic data, curation, and testing system 801 can include an on-demand testing environment 802 and a submission and curation environment 803. A non-limiting embodiment of the testing environment 802 is illustrated in FIG. 10, with a genetic data storage server collocated with a remote client that is in communication with a genetic data interpretations server for conducting the genetic test. A payer party 804 can pay for a genetic test to be conducted on a genome or portion of a genome of a patient 809, as illustrated by arrow 805. Although payer party 804 is shown as an insurer in FIG. 1, one of skill in the art will understand that any other party can pay for the genetic testing. The genetic data, curation, and testing system 801 can conduct the genetic test as described and return results of the genetic test to doctors or clinicians 807, as illustrated by arrow 806. The clinicians 807 can provide the results to patients 809, as illustrated by arrow 808. One of skill in the art will understand that in certain circumstances, the patients 809 can request a genetic test and receive the results directly, without the need to include clinicians 807.

In the submission and curation environment 803, a payment application can account for payments to parties that provided work and data that go into the genetic test. For example, an independent researcher 810 may submit genetic variants and the estimated effects of the genetic variants to a genetic information database, as illustrated by arrow 811. Universities 812, or other researchers, can also submit genetic variants to the genetic information database as illustrated by arrow 813. Independent curators 814 can review the genetic variants and provide curation information as illustrated by arrow 815. The genetic variants and curation information can be used by a test developer in developing a first genetic test 816. Additional submitters, such as corporations 818 can also submit genetic variant information that can be used in developing the first genetic test 816 or a second genetic test 820, as illustrated by arrow 819. In certain embodiments, submitters can be provided a royalty each time a genetic variant is viewed by a subscribing party that pays a subscription to view data in the database. The independent curators 814 can also curate the information submitted by the corporations 818, as illustrated by arrow 819. One of skill in the art will understand that the “independent researcher,” “universities” and “corporations” labels in FIG. 1 are for illustrative purposes only. Any party can submit genetic variants that can be used in any genetic test, as described.

The fees paid by the payer party 804 can be accounted for by the payment application for conducting the genetic test 816, illustrated by arrow 805. As described, the payment application can use an algorithm to distribute royalties for conducting the genetic test, as illustrated by arrow 823. Royalties can be paid to the submitters that submitted the variants used in the genetic test 816, including independent researchers 810, universities 812, and any other submitters, as illustrated by arrows 824 and 825. The payment application can also account for royalty payments to the independent curators 814, as illustrated by arrow 826. In certain embodiments, the curators 814 can receive royalties when a genetic test using a variant curated by the curators 814 is conducted. Alternatively, the curators 814 can receive payment each time they curate a variant, or each time a variant curated by the curators 814 is viewed by a subscribing party without earning royalties from the genetic test 816. In certain embodiments, the curators 814 can be given the option of receiving a payment for curation or royalty payments for the genetic test 816, or can elect to receive a smaller payment for curation services as well as some royalties.

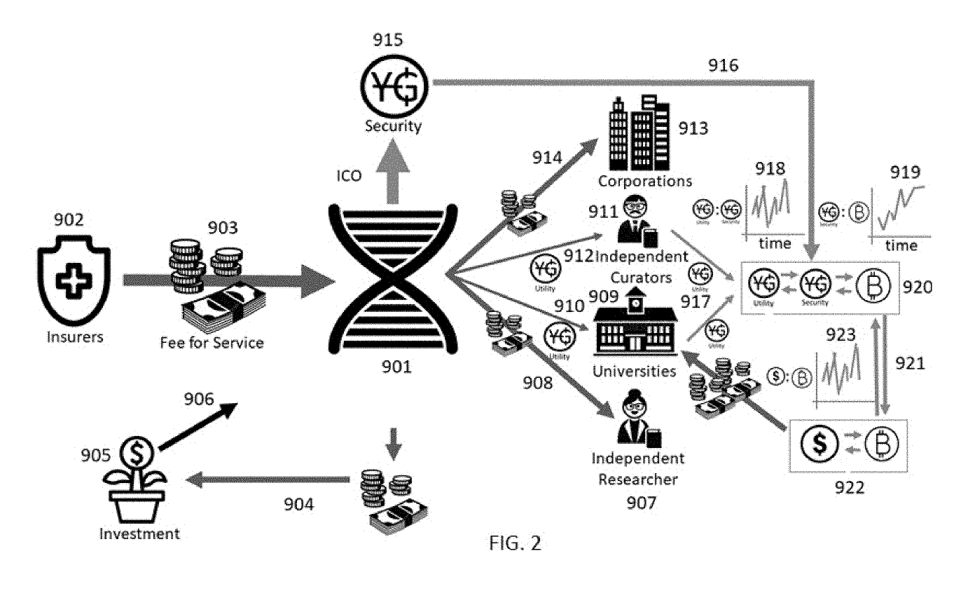

FIG. 2 illustrates an embodiment that uses currency as well as a cryptocurrency to account for payment to each party. Payer parties 902 can pay into the genetic data, curation, and testing system 901 for services, such as conducting a genetic test, as illustrated by arrow 903. Generally, the payer parties 902 will pay in a fiat currency. The system can, in certain embodiments, invest the amount received from the payer parties 902 in an investment 905 as illustrated by arrow 904. The returns on the investment 906 can be used to provide value for the cryptocurrency. Each time a genetic test is conducted, royalties can be paid to the submitters, curators, and test developers, as described. For example, an independent researcher 907 and university 909 that submitted variants used in the genetic test can receive royalties each time the test is conducted, as illustrated by arrows 908 and 910. Curators 911 that curated the variants used in the genetic test can receive royalties as well, as illustrated by arrow 912. Corporations 913 that developed the genetic test can receive payments as illustrated by arrow 914. The payments can be made in a fiat currency, utility tokens, or security tokens. In FIG. 2, the payments to the curators 911 and universities 909 are shown as utility tokens, while the payments to the corporations 913 and independent researcher 907 are shown as fiat currency. However, any of the payments can be made by any payment option.

Fiat currency is a currency that is issued and backed by a government but is not backed by any commodity, such as US dollars. A utility token is a token that gives the user access to a product or services at a discounted rate. For example, a utility token issued by the system illustrated in FIG. 2 could entitle the user to view variants or curation information and develop genetic tests. A security token is a cryptocurrency that is held as a share in the system. The security tokens have value due to the value of the overall system and can be exchanged in a marketplace for money or other commodities. For example, the curators 911 and universities 909 of FIG. 2 receive utility tokens. The utility tokens can be used by the receiving parties, they can be sold to third parties that will then use the utility tokens, or can be exchanged for a security token in a marketplace. In FIG. 2, the curators 911 and universities 909 exchange the utility tokens in a marketplace 920 for security tokens 915, as illustrated by arrows 917. The exchange rate for utility tokens to security tokens 915 can be free floating, as illustrated by graph 918. The security tokens 915 can also be bought or sold in the same marketplace 920, or through any other marketplace or transaction. In FIG. 2, the security tokens 915 are exchanged for a cryptocurrency, such as Bitcoin. The exchange rate between the security tokens 915 and cryptocurrency can also be free floating, as illustrated by graph 919. The system owner can distribute the security tokens 915 into the marketplace 920, as illustrated by arrow 916. The cryptocurrency can then be exchanged in a second marketplace 922 for a fiat currency, as illustrated by arrows 921, at a free-floating exchange rate, as illustrated by graph 923.

One of skill in the art will understand that any of the transactions described can be conducted with any method of payment. Further, combinations of fiat currency, utility tokens, and security tokens can be used. In certain embodiments, the individual users may select how payment is received.

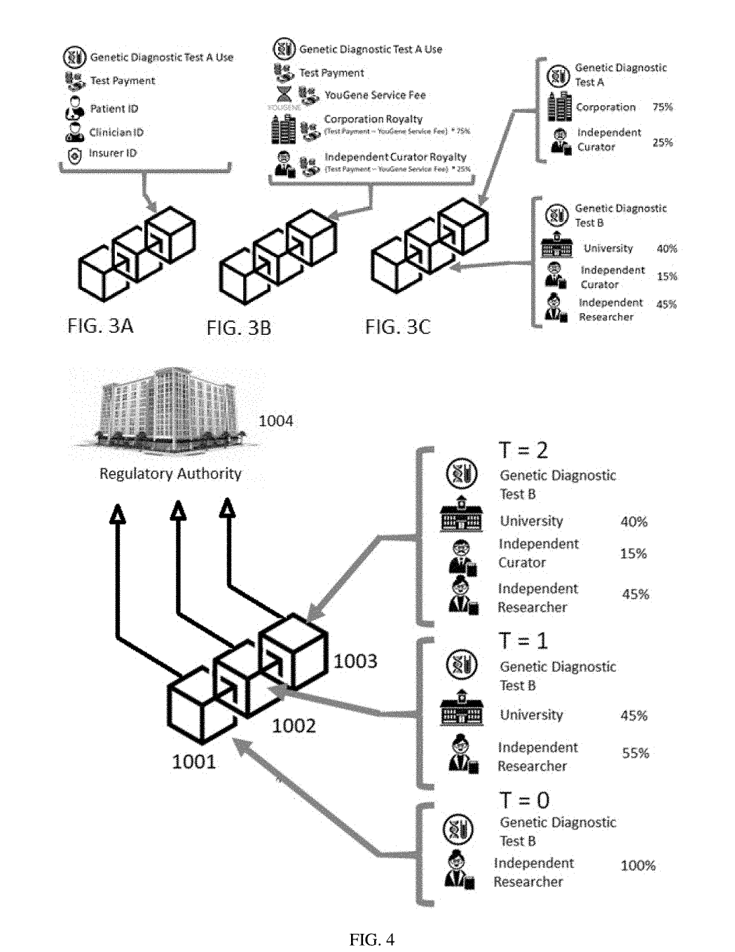

To account for and keep track of payments throughout the system, the system can use one or more blockchains, as illustrated in FIGS. 3A-C. FIG. 3A is a genetic test transaction block chain ledger accounting for payments received by payers for conducting a genetic test. FIG. 3B is a royalty distribution block chain ledger accounting for royalty distributions. FIG. 3C is a dynamically evolving genetic test contribution and ownership allocation blockchain.

As illustrated in FIG. 3A, each time a genetic test is conducted, the blockchain ledger accounts for payment from the payers to the system. Each block in the blockchain can include the amount paid, a patient ID, clinician ID, and insurer ID. Any personal information for any party can be encrypted to secure privacy. All users of the system can view the ledger to ensure that all payments are properly accounted for. Each time the genetic test is conducted, a new block is added, informing all users that the genetic test was conducted and payment has been accounted for.

As illustrated in FIG. 3B, each time the genetic test is conducted, the blockchain ledger can account for disbursements of royalties to all parties entitled to royalties. For example, each block in the blockchain of FIG. 3B can include the genetic test, the payment received, the amount retained by the system owner, and the royalties to each other party. All users can view the royalty distribution blockchain ledger to ensure that all royalties are properly disbursed.

As illustrated in FIG. 3C, for each genetic test, the royalty disbursement can be dynamically evolving. For example, when the test is created at the first block, the royalties are allocated to the corporation that submitted the variants, as well as the independent curator that validated the information used in the genetic test. The relative percentage of the royalties received can be set by an algorithm, as described, to provide an approximation of the value of each party's contribution. A second genetic test is shown in the second block, with royalties allocated to an independent researcher and university that submitted the genetic variants, as well as to the independent curator that validated the information used in the genetic test. Any number of blocks can be included in the ledger for any number of genetic tests. The relative percentage of the royalties received can be set by an algorithm, as described, to provide an approximation of the value of each party's contribution.

As described, the royalty distribution blockchain ledger can be dynamically evolving based on the actions of the users. FIG. 4 provides an illustrative example. In the illustration of FIG. 4, at time T=0, an independent researcher identifies a genetic variant that correlates with a disease and submits the findings along with supporting evidence to the system, creating a new, though unvalidated, genetic test. With the creation of a genetic test, a first block 1001 is created in the blockchain ledger. The block indicates that for the genetic test, the independent researcher is entitled to 100% of the royalties. At time T=1, an unrelated submitter, such as a university, submits additional genetic variants that, when used with the independent researcher's contribution, improves the diagnostic capability of the genetic test. A second block 1002 is created indicating that the university is now entitled to a percentage of the royalties from the genetic test that uses all of the genetic variants. At time T=2, an independent curator validates the findings of the independent researcher and the university increasing the validity of the genetic test, which in turn, increases the value of the genetic test. A third block 1003 is created, indicating that the independent curator is now entitled to a portion of the royalties for conducting the genetic test. The relative distribution of royalties is adjusted to account for the relative contributions of each party to the value of the genetic test.

Although illustrated in FIGS. 3C and 4 as having a single party submit the variants and develop the genetic test, one of skill in the art will understand that the submitter and test developer may be different. The blocks in the blockchain ledger can indicate the relative royalty distributions to each party that contributes to the test, whether as a submitter, a test developer, or an independent curator.

Using blockchain ledgers to keep track of the transactions and payment distributions of the system provides significant advantages. The blockchain ledger provides full transparency and real-time access to current royalty allocations as the genetic research community contributes to a genetic test. The blockchain ledger also provides immutable records of changes in royalty allocations over time. The separate blockchain ledgers also provide “selected transparency” between the different blockchain ledgers for users to ensure that revenues and royalties are properly allocated and accounted for. As described, the personal health information or other identifying information of the users can be encrypted to ensure privacy where appropriate.

The blockchain ledgers described can be separate from the databases containing the genetic information and genetic variants. As described, the patient genetic information can be stored in a genetic data storage server. The genetic variants and curation information can be stored in a centralized database, or stored in separate databases, such as a genetic information database and a curation database. In certain embodiments, a single genetic information database can include the variants and their effects, as well as curation information submitted by the curators. One non-limiting example of a centralized database is the Interplanetary file system, although any centralized database can be used. The blockchain ledgers can be used only to store transaction data, such as payments accounted for and royalty distributions. The blockchain ledgers can be stored on Ethereum, Qtum, NEO, HyperLedger Fabric, or any other blockchain platform. A dApp, or decentralized application, can run on the blockchain ledgers to create the blockchains. The blockchains themselves are distributed ledgers, which are a consensus of replicated, shared, and synchronized digital data geographically spread across multiple computers, sites, countries, or institutions. Because the blockchain ledgers are shared across multiple computers and sites, each user has access to all of the information in the blockchain, ensuring full transparency. The transparency, as well as the royalty distribution, incentivizes work by independent researchers, curators, and other parties, increasing the value of the genetic tests by allowing the parties to monetize their contributions.

As illustrated in FIG. 4, in certain embodiments, one or more regulatory authorities 1004, such as the FDA, can have access to the blockchain. The regulatory authorities 1004 can provide oversight of the information used in genetic tests, as well as information in the genetic information database. For example, the FDA can recognize a genetic variant database if certain criteria are met. The criteria can include submission of the information in the genetic variant database, policies and standard operating procedure in line with FDA guidance, and maintenance of the recognition. The FDA may require that the variants be accompanied by metadata, including information about the analytical performance of the test used to detect the variants, including the number of independent laboratories and/or studies reporting the variant, the name of the laboratory(ies) that reported the variant, the name of the test used to detect the variant, details of the technical characteristics of the test that was used, variant characteristics (which could include but is not limited to, patient ethnicity, zygosity, phasing, and segregation), additional information about the context in which the variant was detected (which could include but is not limited to, variant allele frequency, tumor only versus tumor-normal matched sequencing, cellularity). For cases in which multiple genetic variants factor into determining the overall risk of developing a disease or condition, database administrators may be required to include any multivariant or polygenic scoring methods used in the metadata. Access to the blockchain can allow the regulatory authorities 1004 to determine the number and identity of parties that submitted variants, as well as the number and identities of the curators. The regulatory authorities can also have access to assertion data used in asserting the effects of the genetic variants, including protocols that incorporate multiple lines of scientific evidence, where available, and appropriately weigh each line of evidence; that use a tiered system of assertions (e.g., pathogenic, likely pathogenic) and adequately describe the meanings of each tier; that incorporate unique details of the gene/disease or condition being evaluated, where available or applicable; and that are publicly available. Where required, the system can allow the regulatory authorities 1004 to “spot-check” the information and assertions in the genetic information database. The blockchain can include a link to the location of the metadata associated with each variant, allowing the regulatory authorities 1004 to access the metadata as needed.

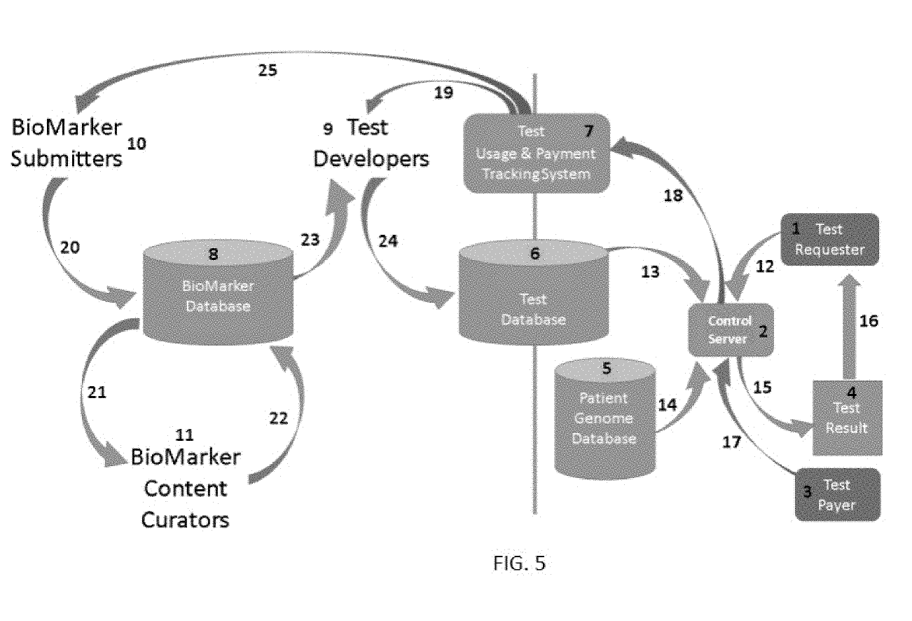

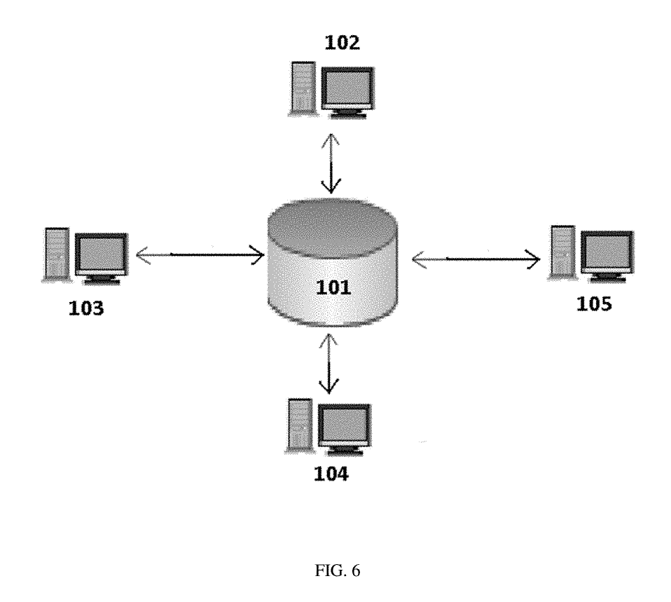

A non-limiting process of genetic test development and conducting genetic tests is illustrated in FIG. 5. One or more biomarker submitters 10 can submit biomarkers to a genetic information database 8, as shown by arrow 20, along with details of the biomarkers, such as the particular variants submitted, estimates of the effects of the variants submitted, demographic information on the source of the biomarkers and any other information that may be useful to determining the actual effects of the biomarkers on the health of individuals. As explained herein, in any embodiment, submitters 10 can make submission of the biomarkers either visible or invisible, allowing the submitters 10 to retain proprietary rights in any information submitted.

One or more curators 11 can access the submitted biomarkers from the genetic information database 8, as shown by arrow 21. The curators 11 can access all of the data submitted by the submitters 10. The curators 11 review the submitted biomarkers and any supporting information submitted by the submitters 10 in order to determine the quality of data submitted and whether or not the curators 11 agree or disagree with the information in the genetic information database 8. In any embodiment, the curators can assign a curation score to the biomarkers and associated information based on the quality of the data and supporting evidence. The assessment of the curators 11 can be returned to the genetic information database 8, as shown by arrow 22. The genetic information database 8 is a master variant and co-occurrence database that can be accessed by users. The system can set a minimum quality or curation score for acceptance of the variant into the genetic information database for commercial use. As explained herein, the assessment of the curators 11 can be used in developing new genetic tests, obtaining approval of new genetic tests, and in determining payment to various parties.

Test developers 9 can access the genetic information database 8, as shown by arrow 23, for the purpose of developing new genetic tests based on the biomarkers submitted by the biomarker submitters 10. As explained herein, the access of the test developers 9 can be controlled based on whether the biomarkers in the genetic information database 8 are made visible or invisible. The test developers 9 can develop new genetic tests based on the submitted biomarkers and the quality of data as determined by the curators 11. The quality of data can also be used by the test developers 9 in obtaining any necessary regulatory approval for use of the new genetic tests. After developing a new genetic test, the test developers 9 can submit the new test to a genetic data interpretations server 6, as represented by arrow 24. The data submitted by the test developers 9 can include electronic instructions that can be carried out by the system in order to conduct a genetic test.

Once a genetic test has been created and submitted to the genetic data interpretations server 6, the genetic test can be used by patients or clinicians. A patient 1 can have all or part of the patient's genome sequenced and submitted to a genetic data storage server 5. As explained herein, all tests submitted to the genetic data interpretations server 6 can be run on the genomic information stored in the genetic data storage server 5, allowing multiple genetic tests to be conducted from a single sequencing of a patient's genome.

A clinician or patient 1 can order a genetic test to be conducted on genetic information in the genetic data storage server 5, as represented by arrow 12. The request to conduct a genetic test can be made to a control server 2, or any other application. The control server 2 or other application can receive the request for a genetic test from the clinician or patient 1, and can retrieve the instructions for conducting the genetic test from the genetic data interpretations server 6, as represented by arrow 13. The control server 2 can also retrieve the particular patient's genetic information from the genetic data storage server 5, as represented by arrow 14, and as explained herein.

The system can conduct the genetic test and generate a test result 4, as represented by arrow 15. The test result 4 can be transmitted to the requesting clinician or patient 1, as represented by arrow 16. A payer party 3 can make a payment to the system for conducting the genetic test, as represented by arrow 17. The control server 2 can account for the payment from the payer party 3, and transmit the payment information to a payment application 7, as represented by arrow 18. The payment application 7 can account for the payment received. The payment application 7, or any other application, can then distribute payments to the original biomarker submitters 10, as shown by arrow 25, as well as the test developer 9, as represented by arrow 19. The system can determine the amounts due to each party, as well as the system owner, based on the contribution of each party to the genetic test, as explained herein.

FIG. 6 shows an overview of the system including the curated database. The system 101 can be accessible by four different types of users. Although shown as a single database in FIG. 6, the system 101 can include any number of databases, servers or other components, as explained herein. One or more submitters 102 can submit information concerning genetic variants to the system 101. The information submitted can include the variants discovered or studied by the submitters 102 as well as estimates of the effect of the variants. Curators 103 can view information submitted by the submitters 102, and determine whether the curators 103 agree or disagree with the information submitted by the submitters 102, or whether there is not enough data presented to make a decision. Based on the opinions of the curators 103, the system 101 can generate a curation score for each variant submitted and curated. Test developers 104 can access some or all of the variant information that has been submitted, along with the curation scores, and develop new genetic tests based on the variants. Patients or healthcare providers 105 can prescribe a genetic test developed by the test developers 104, and, after submission of a patient genetic sample, obtain the results of the genetic test. The system 101 can account for payments from the patients or healthcare providers 105 and disburse payments to the test developers 104, submitters 102, and curators 103 based on the use of the genetic test that has been developed, as described herein.

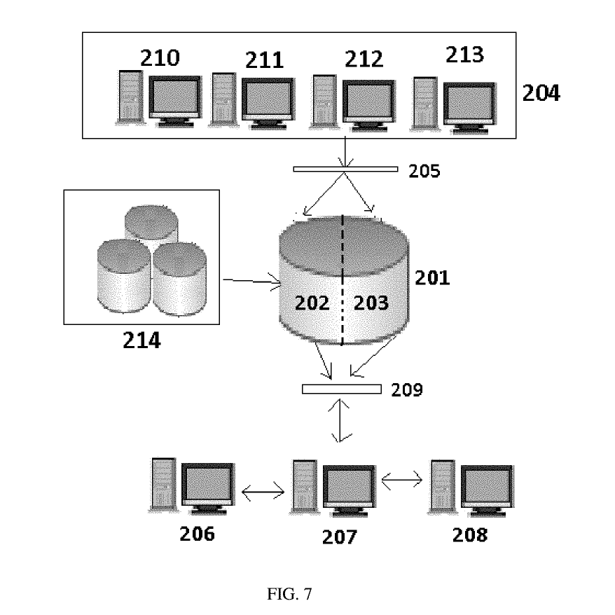

FIG. 7 illustrates the test submission and curation functions of the system. Submitters 204 can submit variant information to a genetic information database 201 through a submission application 205. In any embodiment, the submitters 204 can include parties such as researchers 210 submitting published or unpublished information, clinical labs 211, expert groups in genetic research 212, or patients 213 through Genome Connect, Free-the-Data or other such programs. In any embodiment, the system may also automatically receive information from one or more linked databases 214, such as the ClinVar database, OMIM, InSiGHT, or any other database that can be linked to the system as described herein. In order to protect the proprietary nature of the research and development that goes into determining the variant information, the submission application 205 can allow for both visible submissions, shown as visible submission portion 202 in FIG. 7, and invisible submissions, shown as invisible submission portion 203 in FIG. 7. Information in the visible submission portion 202 of the genetic information database 201 can be accessible by any user of the system. Information in the invisible submission portion 203 can be tightly controlled, such that only the original submitter 204, other users authorized by the submitter 204, and curators can view the information. Because other users, such as other submitters and test developers cannot view the invisible information, the proprietary nature of the invisible information is protected. Although shown as separate sections of the genetic information database 201, one of skill in the art will understand that the visible submission portion 202 and invisible submission portion 203 need not be physically separated, and in any embodiment a single database can house both the invisible and visible submissions. In any embodiment of the invention, the information submitted by the submitters 204 can include the location of the variant in the genome, the type of variant, such as a copy number variation, SNP, translocation, duplication, deletion, addition, or any other genetic variants known in the art. The information can also include the effects of the variant on a subject, at least in the opinion of the submitter 204. In any embodiment, the effects of the variant on a subject can be determined on a scale of pathogenicity. The ClinVar database uses a five-level scale of pathogenicity, including determinations that the variant is benign, likely benign, of uncertain significance, likely pathogenic or pathogenic. In any embodiment, the information submitted can also include the origin of the allele, the gender of affected patients, the age range of affected patients, the ethnicity of affected patients, and the prevalence of the variant across any of the demographic groups described. An example of a common database for collection and study of genetic variants is the ClinVar database. Any of the information included in the ClinVar database can also be submitted to the systems described herein. In any embodiment, information that a user submits to the ClinVar database can automatically be submitted to the system described herein. For example, a user may select an option within the ClinVar database that automatically transmits the submitted information to the described system. Rankings and other information provided by the ClinVar database can also be transmitted and used in the payment algorithms, as described herein. The ClinVar, or any other open database can provide submitters with such an option for each submission, or present a submitter with the option to have all information transmitted to the present system every time a submission to the ClinVar database is created. Because the ClinVar database is publicly accessible, any information submitted through the ClinVar or other open database would necessarily be visible.

The user can also submit supporting evidence for the assertion of pathogenicity. For example, the user can submit how the assertion is made, such as through research, clinical testing, literature or modeling of the effects of the variant and the actual evidence used to make the assertion. The curators can use the information, as explained herein, in curating the variants. The curators can determine the strength of the evidence submitted on the likelihood of a correct assertion of pathogenicity. For example, population data submitted showing a frequency of the variant too high for a particular disorder would be strong evidence that the variant is benign. Population data showing that the prevalence of the variant is increased in affected populations would provide strong evidence that the variant is pathogenic. Computational data or modeling data showing changes in amino acids due to the variant in particular regions would provide evidence of pathogenicity. Functional data, such as studies showing the effects of the variant can be submitted. Segregation data, showing the degree of co-segregation of the variant with multiple affected patients can also provide evidence of pathogenicity. One of skill in the art will understand that any data that supports the assertion of pathogenicity can be included, such as de novo data, allelic data, and data from other databases. In any embodiment, curators can be granted access to all available data in determining the curation score for a particular variant, including data submitted by submitters other than the submitter of the variant being curated. In any embodiment, the curators can be limited to only data submitted by the particular submitter of the variant being curated, or limited to only data available at the time the variant was submitted.

As explained herein, the system allows for curation of all information submitted to the genetic information database 201 by qualified curators 206, 207, and 208. Although shown as three curators in FIG. 7, on of skill in the art will understand that any number of curators can be included. Additional curators, curating the same variant data, will provide additional confidence in the accuracy of that data. The curators, 206-208 can view the submitted variant information and determine whether the effects of the submitter variants are correct. In order to ensure curation of all submitted variants, the curators 206-208 can be allowed to view information in both the visible submission portion 202 and invisible submission portion 203 of the genetic information database 201. In order to protect the proprietary nature of the information in the invisible submission portion 203 of the genetic information database 201, the curators 206-208 may be required to sign an agreement that that the curators 206-208 will not disclose any information designated as invisible. The curators 206-208 can access the information in the genetic information database 201 through a curation application 209. In any embodiment, the curation application 209 can be configured to inform the curators whether the information is visible or invisible. Because the curators 206-208 can access both visible and invisible information, the entire genetic information database 201 can be considered a curated database, regardless of whether the data submitted is made publicly available.

In any embodiment of the invention, the curators 206-208 may be able to rate each variant in the genetic information database. In any embodiment, the curators 206-208 may be allowed to give each variant a curation score, reflecting the confidence of the curators 206-208 in the information submitted. One of skill in the art will understand that the curation score may be provided on any scale. In any embodiment the curation score can be provided on a scale of 1-10. In any embodiment of the invention, the curators 206-208 may be able to provide a separate score if the curators 206-208 believe that there is not enough data to provide a scaled curator score. The curation score can be utilized by the system in calculating the payment algorithms, as described herein. Because multiple curators can curate the same variant, in any embodiment, the curation score can be based, at least in part, on the number of curators that have curated the particular variant. For example, if two curators agree on the effect of the variant submitted by the submitter, the curation score can be higher than if only a single curator has curated the data. In any embodiment of the invention, the curation score can be an average of the scores provided by each curator that has curated a particular variant.

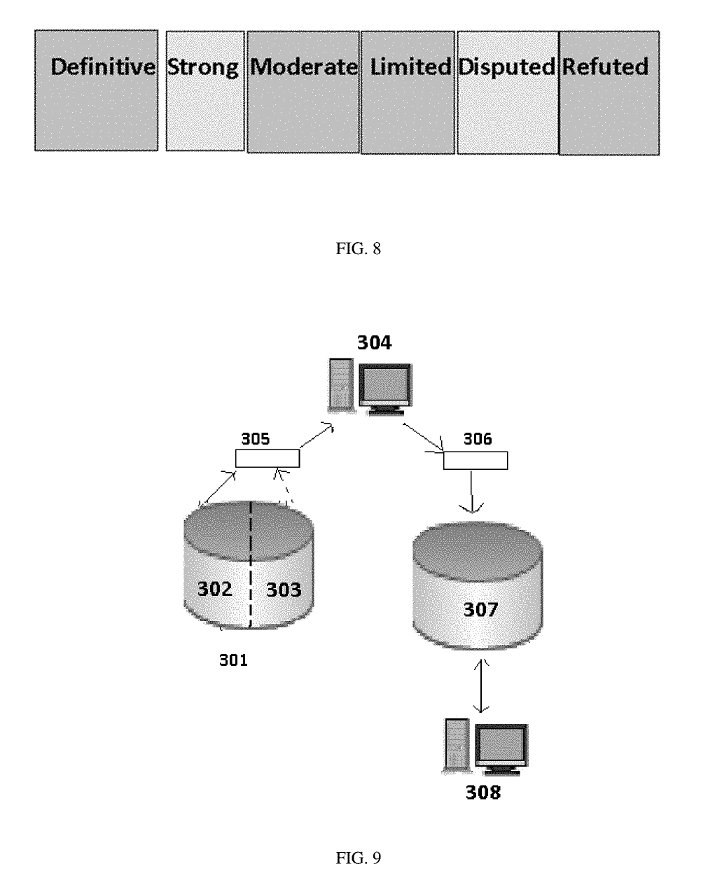

As shown in FIG. 8, the curators 206-208 of FIG. 3 can assign a score based on any evidence available to the curators 206-208. In FIG. 4, the curators 206-208 assign a score corresponding to an assertion of pathogenicity by the submitters ranging from definitive evidence supporting the assertion to evidence refutes the asserted pathogenicity. The score can be based on all evidence available to the curators 206-208, including the type of evidence submitted by the submitters. The scores can be based on data such as the number of probands with clinically associated variants, the amount of functional data available, the number of publications describing patients with the variants, the amount of time since the first publication of the variant, and the strength of any refuting evidence.

In any embodiment of the invention, the curators 206-208 can be allowed to see the scores presented by each of the other curators 206-208. The curators 206-208 can determine whether their assessment matches with the assessments provided by each of the other curators 206-208. In any embodiment, the curators 206-208 may be allowed to score each of the other curators 206-208 on how confident the curators are in the curation scores provided by each of the other curators 206-208. Payment to curators 206-208 can be affected by the curator peer scores.

Any of the described systems can be used by a requester to request curation of any submitted data. The requester can submit data to the genetic information database. The requester can then request that the data be curated through a curation request application. The curation request application transmits the curation request to the curation application 209 and the variant data is curated by the one or more curators 206-208. The one or more curators 206-208 provide a curation report, indicating the level of confidence the curators have in the submitted data. The curation report can be transmitted back to the curation request application and to the requester. As such, the requester can obtain curation of variant data with or without actually making a submission to the system.

FIG. 9 illustrates the process of generating and running a genetic test using the information in the genetic information database. A test developer 304 can access the variant data in the genetic information database 301 through a test developer application 305. In any embodiment, only information in the visible portion 302 of the genetic information database 301 can be available to the test developer 304. In any embodiment test developer application 305 can determine if the test developer 304 is also a submitter of invisible data in the invisible submission portion 303 of the genetic information database 301. In any embodiment, the test developer application 305 can allow a test developer 304 to access the invisible data in the invisible submission portion 303 of the genetic information database 301 only if the test developer 304 was the submitter of the invisible data. That is, if a submitter of invisible data is also a test developer, the submitter of the invisible data can use the invisible data in developing a test. The test developer 304, however, would not be allowed access to invisible data in the invisible submission portion 303 of the genetic information database 301 that is submitted by a different party. In any embodiment of the invention, a submitter of invisible data can allow certain other parties to access the invisible data through a test developer application 305, such as parties to a joint research agreement or other agreement.

The test developer 304 can develop a new genetic test using data in the genetic information database 301. The new genetic test can be submitted to a testing platform 307 through a test submission application 306. The testing platform 307 can then be used by patients or healthcare providers 308 to conduct a genetic test for a patient on a genetic sample submitted by the patient, as explained herein. The testing platform 307 can conduct the genetic test and provide the results back to the patient or healthcare provider 308. Although shown as two different platforms in FIG. 9, one of skill in the art will understand that the genetic information database 301 and testing platform 307 can be housed on the same server, platform or cloud. In any embodiment, the created genetic test can be kept as proprietary information. Any of the algorithms or instructions used in the test can be restricted so that other parties cannot access the information, retaining the proprietary nature of the new genetic tests. In any embodiment, the system can allow only certain parties to view the instructions or algorithms used in the genetic test, such as regulatory bodies or any party specifically granted access by the test developer 304.

### Genetic Testing Platform

One embodiment of the genetic testing platform, described in FIG. 9, is illustrated in FIG. 10. In any embodiment, the genetic testing platform can include a control server (CS) 416 that can be hosted on the web, cloud, server or any other location. The CS 416 is capable of exchanging information between one or more databases located on the same or different servers. In any embodiment, the CS 416 can be an application running on one of the servers.

In one embodiment, a remote client application (RCA) 415 owned by a first company can also be a web, cloud, intranet or server hosted application. The RCA 415 can be affiliated with the CS 416. Multiple RCA's can exist on the same or separate cloud, intranet, or server. In some embodiments, the RCA 415 can be a temporary application on the remote cloud, intranet or server. In other embodiments, the RCA 415 can be permanent.

The genetic data storage server (GDSS) 410 can be a web, cloud, intranet or server data repository owned and, optionally operated by a second company behind a firewall. In any embodiment, the GDSS 410 can be owned by the same party as any of the other applications, servers or databases. In some embodiments, the GDSS 410 can be operated and maintained by the first company. In other embodiments, the GDSS 410 can be operated by a third party, e.g. second company. The GDSS 410 can contain one or more digital test records. In some embodiments, the digital test records can include genetic test records of patient germline DNA. In other embodiments the digital test records can include other biological test data, such as somatic tumor cell DNA or protein or enzyme information. The GDSS 410 can communicate with the collocated RCA 415, responding to requests from RCA 415 and providing test results. In any embodiment, GDSS 410 can be on the same server, virtual instance, intranet, behind the same firewall, or in the same cloud environment 421, as RCA 415. Collocation eliminates the need to send the sensitive, and very large, digital test results across the internet. In some embodiments the RCA 415 can be embedded as part of the GDSS 410. In other embodiments, the RCA 415 can operate outside of the GDSS 410, while the RCA 415 is collocated with GDSS 410. In some embodiments, the RCA 415 can be located on a different server, virtual instance, intranet, firewall, or cloud environment as RCA 415.

One example of a genetic data storage server (GDSS) is the Illumina® Sequencing and Array Based Solutions system, e.g. BaseSpace. Other genetic data storage servers presently known can include Curoverse, GA Biobank, or any other known biorepository. The GDSS system typically offers the sequencing and storage of genetic data. However, any storage system, biobank, data repository, biorepository, or data commons capable of storing genetic data either in WGS, WES or any other known suitable output is contemplated by the invention. In some embodiments, the genetic data storage server can be any HIPAA compliant server capable of storing genetic data.

The genetic data interpretations server (GDIS) 417 can be a web, cloud, virtual instance, intranet, or server based data repository. The GDIS 417 can be operated by the first company or by a third party. The GDIS 417 can contain one or more biomarker scripts, with clinical interpretations based on results generated for the biomarker scripts generated by the test developers as illustrated with respect to FIG. 9.

The digital patient information storage server (PISS) 418 can be a web, cloud, intranet, or server hosted data repository. In some embodiments, the PISS 418 can be operated by the first company. In other embodiments, the PISS 418 can be operated by a third party. The PISS 418 can contain one or more patient records. The PISS 418 can communicate with CS 416 and can operate to update, edit or delete patient information.

One or more listeners can be used on any of the data repositories in order to create dedicated server processes for each user, and thereby increase efficiency and decrease memory constraints. In some embodiments, the data can be communicated using JSON or other communication protocol.

The CS 416 can be hosted in a separate cloud environment, intranet, or server 422 as RCA 415. However, in some embodiments, CS 416 can be in the same cloud environment, intranet, or server as RCA 415. In some embodiments, CS 416 and RCA 415 can be located on a single intranet. GDIS 417 and PISS 418 are shown in FIG. 10 as being in a single cloud, intranet, or server 423. In other embodiments, GDIS 417 and PISS 418 can be in separate clouds or servers. In some embodiments, GDIS 417 and PISS 418 can be in the same cloud, server or intranet as CS 416. One of skill in the art will understand that any arrangement of the applications, databases and servers described is within the scope of the invention.

After all the software is installed, a communications portal 401 can be established between the CS 416 and the RCA 415. A second communications portal 402 can be established between the CS 416 and PISS 418. A third communications portal 403 can be established between the CS 416 and GDIS 417. A fourth communications portal 414 can be established between the RCA 415 and GDSS 410. The Communication portals 401, 402, 403 and 414 can be established and maintained via any combination of TCP, UDP, VPN, sockets, OS messaging or equivalent technologies suitable to transmit secure and unsecure information between two collocated or non-collocated software instances.

In any embodiment, a library, DLL, extension or API can be written into the genetic data storage server (GDSS) such as an operator, e.g. Illumina Basespace or any local hosting server, that can be incorporated into the GDSS owner's software that would allow the GDSS owner to run scans within their module by incorporating an outside code. Thus, a GDSS can remain isolated and protected yet receive instructions via the Remote Client described herein. In particular, the embedded software, DLL or API can operate as the Remote Client, communicating with the Control Server, but embedded within another application.

For example, a prescription 404 to test a biomarker can be obtained by the CS 416 from a patient's electronic health records or electronic medical records, or from a health care provider 420. In some embodiments, health services providers can generate prescriptions directly through electronic health records and the prescription can be directly sent to the CS 416. Non-limiting examples of services for generating prescriptions directly through electronic medical records include Allscripts® or Surescripts®. However, any electronic prescription service is contemplated by the invention. In other embodiments, the prescription 404 can be transmitted to CS 416 by the health services provider through a user interface (not shown).

In any embodiment, an environment can be provided that runs open source and/or commercial tools (e.g. Galaxy, GATK, etc.). The environment can provide for deep provenance and reproducibility across all connections and provide a means to flexibly organize data and ensure data integrity. In any embodiment, the invention contemplates means for running distributed batch processing jobs that provide for secure sharing of data sets. The invention also contemplates providing a set of common APIs that enable application and pipeline portability across systems. The invention can be platform and system agnostic. In each instance, the invention can handle storing and organizing large data sets (e.g. BAM, FASTQ, VCF, etc.) and handle storing metadata about files for a wide variety of organizational schema. The invention further provides for an environment where stakeholders such as the genetic data submitter, the genetic test submitter, the prescriber, or control application owner can receive access to virtual machines (VMs) on a private or public cloud thereby eliminating the need to manage separate physical servers. In any embodiment, any of the services described herein including prescription, connections and scripts can be accessed through APIs.

For example, the prescription 404 can be communicated to CS 416. Digital test identification information 405 can be retrieved from the PISS 418 and communicated to the CS 416. The digital test identification information can include information necessary for locating one or more digital test records from GDSS 410, or data that can be used to generate all or part of a patient's genome. The digital test identification information can be sent 406 to RCA 415 for the purpose of locating one or more digital test records from GDSS 410. The digital test records can be retrieved and sent back 407 to the RCA 415. The digital biomarker script constituting the genetic test instructions can be retrieved 408 from the GDIS 417 and sent to CS 416. The CS 416 can send the digital biomarker script 409 to the RCA 415. The script can be responsible for providing instructions to the RCA 415 necessary for the interpretation of the genetic or other biological data in accordance with the biomarker test prescription 404.

In any embodiment, the biomarker test prescription 404 can include any one or more of a biomarker identifier, a patient identifier, a physician identifier, a payer identifier, a test data identifier, and a test data location identifier where one or multiple GDSSs and RCAs are used as described herein.

The RCA 415 can execute the instructions in the biomarker script, operating on the digital test record. The results of the script can be returned 411 to the CS 416. The results of the script can be communicated 412 to the prescriber 419. In some embodiments, the results can be communicated 412 electronically. In other embodiments, the results can be communicated 412 to the prescriber 419 via any possible means of communication. The results of the script can also be archived 413 on the PISS 418.

By collocating the RCA 415 and GDSS 410, a patient's genetic information can be queried, analyzed, and the results transmitted, without the need for transmitting the patient's actual genome across the internet. In any embodiment, the RCA 415 and GDSS 410 can be remote from each other, and the patient's genetic data can be communicated between the RCA 415 and GDSS 410. In other embodiments, PISS 418 is unnecessary. The specific patient information can be obtained directly from the prescriber or health care provider 420 and transmitted to CS 416.

In certain embodiments, the RCA 415 can iteratively search the genetic information contained in the GDSS 410 for biomarkers and genetic variants associated with a given condition in accordance with instructions from the GDIS 417. Known search engines and parser algorithms such as BLAST, BioJava (http://www.biojava.org/wiki/Main Page) or BioParser (http://bioinformatics.tgen.org/brunit/software/bioparser/) can be used to search the diagnostic information for relevant proprietary biomarkers, as well as any other algorithms known in the art.

Although a single embodiment is shown in FIG. 10, one of skill in the art will understand that other arrangements of the applications, servers and databases can be used to execute a genetic test. Any arrangement is within the scope of the invention.

### Genetic Data Scoring

As described herein, the system can account for payments to test developers, curators, and submitters of the variant data used in the test development each time a particular genetic test is conducted. The payments can be calculated based on an algorithm that accords certain point values to each of the variants used in the genetic test, which can then be used to determine the payments due to the test developer and the data submitters.

As described herein, in any embodiment, the variant information submitted by a submitter can include the variant location in the patient genome, the submitter's estimate of the pathogenicity of the variant, and a phenotype associated with the variant. In any embodiment, the information submitted can also include the origin of the allele, the gender of affected patients, the age range of affected patients, the ethnicity of affected patients, and the prevalence of the variant across any of the demographic groups described. An example of a common database for collection and study of genetic variants is the ClinVar database. Any of the information included in the ClinVar database can also be submitted to the systems described herein. In any embodiment, information that a user submits to the ClinVar database can automatically be submitted to the system described herein. For example, a user may select an option within the ClinVar database that automatically transmits the submitted information to the described system. Rankings and other information provided by the ClinVar database can also be transmitted and used in the payment algorithms, as described herein. The ClinVar, or any other open database can provide submitters with such an option for each submission, or present a submitter with the option to have all information transmitted to the present system every time a submission to the ClinVar database is created. Because the ClinVar database is an open database, any information submitted through the ClinVar database will necessarily be a visible submission. However, as described herein, invisible data can also be submitted, wherein the invisible data is not made publicly available, and can only be used by the original submitter or any party identified by the original submitter as having access to the data.

In any embodiment, the information within the system can be made available to researchers or other parties. In any embodiment, the information within the system can be made available to the other parties through a variant usage application, which can control and track the usage of the genetic variant information. In any embodiment, users of the information in the genetic information database can be required to pay into the system as subscribers, which can be used to pay the original submitters, as described herein. In any embodiment, the cost of using the information can be varied based on the type of user or the purpose of the information. For example, the information may be made free to non-profit researchers, while for-profit users would need to pay. One of skill in the art will understand that several permutations of payment for use of the system can be created. For example, doctors may be considered either commercial users or non-commercial users. In any embodiment, the user may be required to disclose whether the use of the genetic information is for commercial purposes. A user that intends to use the information for commercial purposes can be considered a commercial user, and be required to pay. A user that does not intend to use the information for commercial purposes can be considered a non-commercial user, and can be provided some or all of the information without payment. In any embodiment, all users may have to pay a fixed amount for usage of the information. In any embodiment, all users may access the visible information freely, and payment to submitters can be accounted for after a genetic test has been conducted, as described herein.

As explained herein, based on the information submitted to the system by submitters, a test developer may be able to identify certain variants that would lead to a particular phenotypic outcome or disease, or an increased chance of a phenotypic outcome or disease. One of skill in the art will understand that the test developer may be able to create a genetic test based on the information in the system. As described herein, the systems and methods disclosed can be used for receiving genetic test instructions and for carrying out genetic testing. As such, in any embodiment, a test developer may use the systems and methods described herein to make a genetic test available to the public. In any embodiment, a test developer may be required to agree to use the described systems for any genetic test created based on the variant information within the system. In any embodiment, the test submission application, described with reference to FIG. 9, can verify the source of the genetic variants used in the genetic test.

In the system and methods described, the submitting parties can be paid for the usage of the information submitted to the genetic information database, regardless of whether that usage results in a genetic test. Additionally, in any embodiment, the submitting parties can be paid for any information submitted, such as a nominal fee for submitting information.

As described herein, the system can receive genetic test instructions and carry out genetic testing. In any embodiment, the system can calculate and account for a payment to test developers and the original submitters each time a particular genetic test is prescribed or carried out, as illustrated in FIG. 11. A prescriber or user 502 may select a genetic test that is part of the described systems. Payment from a payer party for conducting the genetic test can be accounted for by the system 501, as represented by arrow 505. The price of the genetic test can be set by any method known in the art. For example, the price of the genetic test can be set by the test developer 503. Alternatively, the price of the genetic test can have some pre-set value, or the price can be determined through any type of marketplace.

As described herein, the system can receive genetic test instructions and carry out genetic testing. In any embodiment, the system can calculate and account for a payment to test developers 503, the original submitters 504, and optionally the curators, each time a particular genetic test is prescribed or carried out, as illustrated in FIG. 11. A prescriber or user 502 may select a genetic test that is part of the described systems. Payment from a payer party for conducting the genetic test can be accounted for by the system 501, as represented by arrow 505. The price of the genetic test can be set by any method known in the art. For example, the price of the genetic test can be set by the test developer 503. Alternatively, the price of the genetic test can have some pre-set value, or the price can be determined through any type of marketplace.

Each time a particular genetic test is conducted, the system can account for payments made to the test developer 503, as represented by arrow 506, and to the submitters 504 that submitted the original variant information used in the genetic test, as represented by arrow 507. The algorithms used to determine the proportional amounts paid to the test developer 503 and original submitters 504 are described herein.

Based on the data submitted by a submitter, a variant score can be calculated by the system for each variant submitted. One of skill in the art will understand that many methods of calculating a variant score can be used and the examples provided herein are provided for illustrative purposes. In any embodiment, a variant score can be calculated only for variants that are made visible by the original submitters, with invisible submissions automatically receiving a variant score of 0. In any embodiment, invisible submissions can still result in variant scores, wherein the variant score is only used by the algorithms if the invisible variant submitter is also the test developer.

In any embodiment of the invention, a variant score can be based on factors such as data quality, submitter credibility, submitter participation in the system, and a time factor. The data quality score can be based on an objective level of confidence in the information submitted to the genetic information database. In any embodiment, the data quality score can be based, at least in part, on a curation score assigned by the curators of the genetic information database. In any embodiment, the data quality score can be independent of the curation score, and can be based on factors such as the amount, type, and quality of data submitted by the submitter. For example, the data quality score can be provided on a scale of 1-5; wherein a variant submitted without any supporting data can be granted a data quality score of 1; a variant submitted with supporting clinical and testing information can be awarded a data quality score of 5; and variants submitted with different types of supporting data can be awarded scores of 2-4, based on the amount and type of data submitted. One of skill in the art will understand that the data quality score can be provided on any scale, as described herein. The ClinVar database utilizes a metric to assess a level of confidence in information submitted, and in any embodiment the same metric can be utilized by the present invention. The ClinVar metric produces a rating of between zero and four stars for the submission. A rating of zero stars is provided if a submitter does not provide an interpretation of the variant, or if there are conflicting interpretations. One star is provided if the user submits an interpretation for the variant, but the interpretation is not supported by any additional submitters or curation. In order to obtain a one star rating, the submitter must document that the allele or genotype was classified according to a comprehensive review of evidence consistent with, or more thorough than, current practice guidelines (e.g. review of case data, genetic data and functional evidence from the literature and analysis of population frequency and computational predictions); include a clinical significance assertion using a variant scoring system with a minimum of three levels for monogenic disease variants (pathogenic, uncertain significance, benign) or appropriate terms for other types of variation; provide a publication or other electronic document (such as a PDF) that describes the variant assessment terms used (e.g. pathogenic, uncertain significance, benign or appropriate terms for other types of variation) and the criteria required to assign a variant to each category; and submit available supporting evidence or rationale for classification (e.g. literature citations, total number of case observations, descriptive summary of evidence, web link to site with additional data, etc.), or be willing to be contacted by ClinVar users to provide supporting evidence. Two stars are provided if multiple submitters meeting the one star criteria provide a single interpretation for a variant. Three stars are provided if the submission is supported by a panel of curators. In order to obtain review by an expert panel, the submitter may request the review. Four stars are provided if the submitter and information are in accordance with certain practice guidelines, which serve to ensure the accuracy of the data. As disclosed herein, in any embodiment, submissions to ClinVar or other databases can automatically be forwarded to the system of the present invention, and in any embodiment the ClinVar confidence rating can be used with or without further review to generate a data quality score. However, one of skill in the art will understand that other metrics for generating a data quality score exist and can be utilized by the present invention. For example, the data quality score can be based solely on the curation score representing a level of confidence assigned by the curators as described herein. Because the curators will have access to all of the information in the genetic information database, the curators can curate and assign a curation score even to invisible information. The invisible, curated, variant information can be used by the submitter in proving the reliability of the test to regulating bodies, such as the FDA.

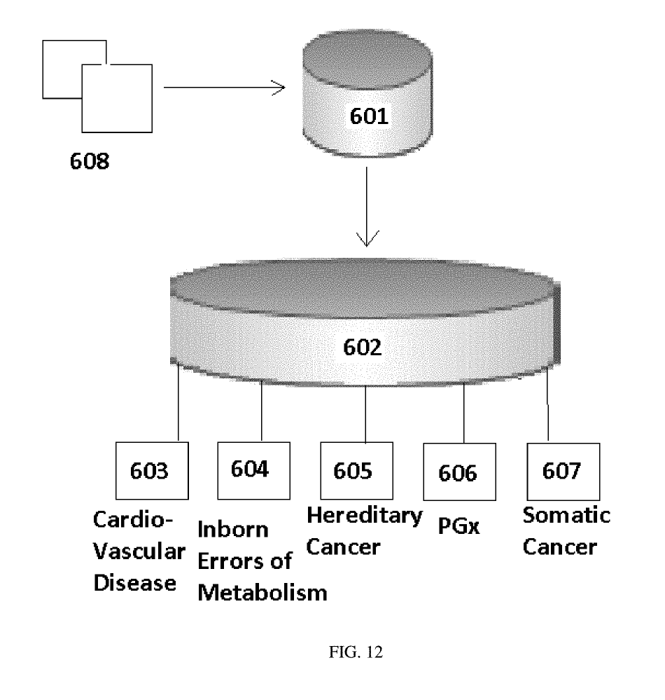

FIG. 12 illustrates one embodiment of the curation process. As described herein, submitters 608, including researchers, clinical labs, or other expert groups, can submit variant information to a genetic information database 601. The genetic information database 601 can be accessed by curators through a curation application 602. In order to ensure that curators are only curating variants in fields of which they are experts, the curators can be separated into curation groups, such as a cardiovascular disease group 603, an inborn errors in metabolism group 604, a hereditary cancer group 605, a PGx group 606 specializing in drug interactions, and a somatic cancer group 607. The curators 603-607 can access the information concerning variants in their particular field of expertise and review the evidence, providing curation scores as described herein. In any embodiment, the expert groups of curators can review the submitted evidence and assertions in conformance with any known standards or guidelines, including the ACMG rules for interpretation of sequence variants. By having multiple curators reviewing the same variant data, as explained herein, in accordance with accepted rules for interpretation, any bias inherent in a particular curator can be reduced.

A submitter credibility score can be based on the level of expertise a particular submitter has with the particular type of variants or pathologies submitted. For example, a party with extensive experience in breast cancer variants may be awarded a higher credibility score for a variant that the submitter believes is associated with breast cancer than for a variant the submitter believes is associated with some other phenotypic outcome. The credibility score can also be based on the level of agreement among other submitters for other variants submitted by the present variant submitter or the curation score for other variants submitted by the present variant submitter.

A participation score can be based on the number of variants submitted by a particular party in total. That is, the first variant submitted by a party may be awarded a participation score of 1, while a second variant submitted by the same party may be awarded a participation score of 2. The participation score encourages parties to submit variant information for additional variants, as the total variant score will increase with each submission. In any embodiment, a submitter may only be awarded participation score points for visible submissions to the genetic information database, in order to further encourage public disclosure of variant information.

The time factor can be used to take into account the order in which multiple submitters submit the same variant. For example, the first submitter to submit a particular variant may be granted a higher time score than a second submitter. The second party to submit the variant may be granted a higher time score than the third submitter, and so on. By scaling the variant score for each submitter based on the order of submission, early submission of newly discovered variants is encouraged. In any embodiment, the time score can be set to 0 for invisible submissions. That is, until a submission is made publicly available, the submitter of invisible data does not get credit for submitting the information first. In any embodiment, if a later visible submitter submits the same data as an earlier invisible submitter, the later visible submitter can be given a time score as if the later submitter was the original submitter.

Eq (1) provides a sample method of a calculation of a variant score.

V=O+Q+C+P+R Eq (1)

In Eq (1), V represents the variant score, O represents a time factor, such as the order of submission of the variant; Q represents the data quality score, C represents the credibility score, P represents the participation score, and R represents the curation score, as described herein. In any embodiment, the time factor can be a multiplier as opposed to an addition, as shown in Eq (2).

V=O*(Q+C+P+R) Eq (2)

In Eq (2) each of the variables are the same as in Eq (1), with the exception that the time factor O is multiplied by each of the data quality score Q, credibility score C, participation score P, and curation score R. In any embodiment, the time factor used in Eq (2) can be set as the reciprocal of the order of variant submission. For example, the first submitter would have a time factor of O=1/1, while the second submitter would have a time factor of O=½, the third submitter would have a time factor of O=⅓, and so on.

In any embodiment, each of the factors included in Eq (1-2) can be scaled in order to emphasize particular desired outcome. For example, the curation score can be provided on a scale of 1-10, while the credibility score can be provided on a scale of 1-5. Such a system would result in greater emphasis on the quality of data submitted than on the party submitting the data. One of skill in the art will understand that any of the scores provided in Eq (1) can be based on any type of scale. Any of the factors leading to the variant score can be omitted in any embodiment. In any embodiment, any of the factors shown in Eq(1-2) can be eliminated. For example, the system need not use the participation score or credibility score. In any embodiment, the data quality score can be eliminated, and the data quality can be reflected by the curation score. Table 1 provides an illustration of one scaling system shown for multiple submitters of two different variants.

TABLE 1

| | Submitter Name | | | |
|---|---|---|---|---|
| | A | B | B | A |
| Invisible/Visible | Visible | Visible | Invisible | Visible |
| Genome ID | rs123 | rs123 | rs123 | rs234 |
| Value | A | A | C | T |
| Submittal Date | Jan. 1, 2014 | Feb. 1, 2014 | Feb. 1, 2014 | Feb. 1, 2014 |
| Disease | Breast Cancer | Breast Cancer | Breast Cancer | Breast Cancer |
| Submission Order | 1 | 2 | 1 | 1 |
| Time Score | 10 | 5 | 0 | 10 |
| Quality Score | 5 | 4 | 0 | 4 |
| Curation Score | 10 | 8 | 10 | 8 |
| Credibility Score | 10 | 5 | 0 | 10 |
| Participation Score | 1 | 1 | 0 | 2 |
| Total Variant | 36 | 23 | 0 | 34 |
| Score | | | | |

In Table 1, the Time Score is calculated as 10 divided by the order of submission. That is, the first party to submit a particular variant is granted 10 points, while the second party is granted 5 points, the third party is granted 3.3 points, and so on. One of skill in the art will understand that any method of calculating a time score is within the scope of the invention, including using a multiplier as the time score as illustrated in Eq(2). Each of the variants shown in Table 1 is shown as a single nucleotide polymorphism, wherein the value refers to the value of a nucleotide at a particular location within the genome. One of skill in the art will understand that the SNPs are provided for simplicity only, and the system can utilize genetic variants of any type, including insertions, deletions, copy number variants, rearrangements, duplications, translocations, inversions or any other type of genetic variant. In Table 1, the data submitted for Genome ID rs123 as C by party B is submitted as an invisible submission. As such, the variant score is automatically set as 0 in Table 1. However, as described herein, a curation score can still be determined for the variant, and the variant can be used by Party B as the original invisible submitter. The payment algorithms set up a micro-attribution royalty framework that protects data submissions and provides control over the submitted data.

In any embodiment, other methods of using the scores provided in Table 1 are possible. For example, the credibility, participation and data quality scores can be multiplied by some value representative of the time score as shown in Eq(2). In any embodiment, the variant score can be calculated based on a first order polynomial utilizing the factors listed in Table 1. One of skill in the art will understand that alternatives to the variant score calculation shown in Eq (1-2) can be used.

Any payment due to submitters for submission of information to the genetic information database can be based on the variant score. Submissions that result in high variant scores can result in increased payment to the submitter as compared to submissions that result in lower variant scores.

As described herein, a user of the variant information can create a genetic test based on the information, and submit the genetic test to the test system. In any embodiment, the system can require that any genetic test be an approved genetic test. An approved genetic test is a genetic test that has received regulatory approval from the appropriate regulating agency, such as the FDA. In any embodiment of the invention, the test developers can use the curation scores from the curation of the genetic information database in obtaining approval of the genetic test.

As illustrated herein, once a test has been created, the described systems can be used to carry out the genetic test and account for payment from a payer party to the appropriate rights holder parties. In any embodiment, the rights holder parties, as used herein, can refer to test creators or variant submitters, as well as owners of any intellectual property rights in any of the information used.

Eq (3) provides a calculation for a total number of variant points associated with a particular genetic variant.

T=Σₖ₌₁ⁿVₖ Eq(3)

wherein T represents the total number of variant points for a given genetic variant, Vₖ represents the variant score for each individual party that has submitted the variant, and k is the total number of parties that have submitted the variant.

The total number of variant points that are awarded for a genetic test can be given by Eq (4):

A=Σₖ₌₁ⁿTₖ Eq (4)

wherein A is the total number of variant points in a genetic test, T is the total variant points for a given variant k used in the genetic test, and n is the number of variants used for the genetic test.

The total value for a genetic test can be given genetic test can be given by Eq (5):

B=A+L Eq (5)

wherein B is the total number of points awarded for a genetic test, A is the total number of variant points for a genetic test, and L is a number of points awarded to the test creator for creating the genetic test.

When a genetic test is conducted, the total variant score can be used to calculate a payment to each of the submitter parties, as shown in Eq (6):

$$S = \frac{U * M}{B} * V \qquad Eq ( 6 )$$

wherein S represents the payment to a submitter for submission of a particular variant, U represents the price paid by the payer party for conducting the test, M represents a system value which is related to payment to the system owner for use of the system, B represents the total number of points awarded for the genetic test, and V represents the variant score for the variant used and submitted by the submitter. One of ordinary skill in the art will understand that the value U*M represents the total cost of the test that is passed on to submitters and test creators, and that is not used as payment to the system owners. One of skill in the art will understand that the total payment to any party can be given by multiplying the value of U*M by the pro rata share of the total points awarded for a given test, whether earned as a variant submitter or a test creator, or both. The pro rata share of points can be given by formula in Eq (7):

$$E = \frac{{\sum }_{{0 \leq i \leq m}_{0 \leq j \leq y}} ( {V}_{i} ⋃ {V}_{j} )}{B} \qquad Eq ( 7 )$$

Wherein E is the pro rata share of variant points or variant royalty rate, T is the total number of variant points, and Vⱼ is the set of all variants used in the genetic test, and Vᵢ is the set of all variants submitted by the submitter. Put another way, the union of variants Vⱼ used in the Test with the variants submitted by particular Variant Submitter Vᵢ, is the total point award for any particular Variant Submitter for any particular Test. Hence, a submitter's variant royalty rate, is the union of the variants that were submitted by the submitter and used in the test, divided by the total variant point award T for the test.

In any embodiment, the system can be configured to pay the system owner for each test conducted, as represented by M in Eq (6). For example, the system can be configured such that the system owner receives some percentage of all test revenue. If the system owner is to receive 30% of all test revenue, then M can be set as 100%-30% or 70%, representing the amount of revenue provided to the submitters and test creators. One of skill in the art will understand that the value M can be set to any number, including 0 in situations where the system owner receives no portion of the test revenue. The system can also account for a payment to the curators that curated the variants used in the genetic test using a similar or different algorithm.

The amount of the payment to the test creator with each genetic test conducted can be given by Eq (8):

D=U−(U*M)−Σ_{b=1}S_{b} Eq(8)

wherein D represents the payment to the test creator, U represents the price paid by a payer party for conducting the genetic test, Sb represents the payment due to a submitter as described in Eq(6), and the variable a refers to the number of submitters that are due to receive payment for the particular genetic test. That is, the value to the test creator for each test conducted is the residual value not paid to the system owner or submitters. One of skill in the art will understand that Eq(8) can be rewritten as Eq(9) to provide the same value.

$$D = \frac{U * M}{B} * L \qquad Eq ( 9 )$$

The test value L can be set at some level representing an amount of work and effort in creating the genetic test. In any embodiment, the test value can be some set number, and each test created can have the same test value. In any embodiment, the test value can vary depending on any number of factors, such as difficulty in obtaining regulatory approval for the test, the complexity of the test, the number of submitters that have submitted variants used in the test, or any other factors. For example, if the created genetic test uses many variants, the test value L can be set higher than for a created genetic test using less variants. In any embodiment, the test value L can be set as some fixed percentage of the total variant points for a test. For example, L can be set as 20% of the total variant points A as defined in Eq (4). One of skill in the art will understand from Eq(4)-(9) that, assuming the same total variant score, a test with a higher test value L will provide a greater proportion of test revenue to the test creator. As such, the value of L can be set at some level representing a proportion of the total test revenue that the system owner wishes to provide to test creators.

One of skill in the art will understand that other algorithms are possible depending on the number of factors used in calculating the variant scores and the relative weights of each of the factors. As a non-limiting example, Eq (10) shows a sample variant score calculation algorithm using only three factors:

V=O*Q*R Eq (10)

wherein V represents the variant score, O represents a time factor expressed as a multiplier, Q represents a data quality score and R represents a curation score. Other algorithms are possible using addition, subtraction, multiplication or division of the variant score factors describe herein, and are within the scope of the invention.

Table 2 provides a sample calculation to the test creators and submitters, using the submission data provided in Table 1. For illustrative purposes, the test creators shown in Table 2 are also genetic data submitters as shown in Table 1.

TABLE 2

| | Test 1 | Test 2 |
|---|---|---|
| Disease | Breast Cancer | Breast Cancer |
| Test Owner | A | B |
| Variants ID (value) | rs123 (A) | rs123 (A) |
| | rs234 (T) | rs123 (C) |
| | | rs234 (T) |
| Test value (L) | 500 | 500 |
| Total Variant Points (T) | 93 | 93 |
| Total Test Points | 593 | 593 |
| Test Price | $600 | $500 |
| System value (M) | 70% | 70% |
| Test Creator Value | $354.13 | $295.11 |
| Total Submitter Value | $65.87 | $54.89 |
| Amount Paid to Each Party | Party A $403.70 | Party A $41.26 |
| per Test | Party B $16.29 | Party B $308.63 |
| | Owner $180.00 | Owner $150.00 |

As illustrated in Tables 1-2, an invisible submission, such as Party B's submission of the data for Genome ID rs123 as C, and as used in the second test of Table 2, receives no variant points, although Party B may use the data in development of a test. Because Party B's invisible submission dose not receive any variant points, the variant does not factor into the algorithms for calculating payment per test. In any embodiment, invisible submissions can be calculated as having a variant score, which may be reduced by some factor in order to encourage public disclosure of the data. For example, a variant submitted as an invisible submission may have a variant score calculated in the same manner as a visible submission, but with a time score of 0. Alternatively, the invisible submission can be given a variant score that is less than what would have been awarded had the submission been visible, such as by dividing the variant score by 2. Any factor for reducing the score of a variant submitted as an invisible submission is within the scope of the invention, such as reducing the variant score by 10%, 25%, 50%, 75% or any factor between 0 and 100%.

As illustrated in Table 2, the majority of the test revenue can be provided to the test creator by scaling the variant scores and test value such that the test value is considerably higher than the total variant score. With Test 1, Party A is a both a submitter and a test creator. The total paid to Party A for each test, as defined by equations 1-5, represents the payment to Party A based on the creation of the test, plus the payment to Party A based on the original submission of the genetic variant information. Conversely, with Test 2, Party B is both a test creator and a submitter. In either case, 30% of the test revenue is provided to the system owner, as shown by the value of M being 70%. In any embodiment, the algorithms can be modified to provide a greater share of the payment to the data submitters, such as be reducing the test value to 100. In any embodiment, the majority of the payment may go to the data submitters, such as by further reducing the test value used. The test value can be set by the operators of the system in order to encourage either data submission or test development by adjusting the relative points awarded to the variants used and the test value.

One of skill in the art will understand that the test creator need not be a data submitter. In any embodiment, the test creator can be any party that has created an approved test, whether or not that party is a submitter, or even in the medical or genetic field. Statisticians, mathematicians or any other party can use the information in the genetic variant database to create a new genetic test, which can be utilized by the system if approved. Because the information in the genetic information database is curated, approving agencies, such as the FDA can be assured of the quality of the data used in developing the test. As such, the system and methods described herein provide for an inducement to non-researchers to study the genetic data provided and create new genetic tests.

The algorithms in Equations 1-5, and illustrated by Table 2, further encourage early submission of data to the system by rewarding the earlier submitters with higher variant scores, which then leads to increased revenue based on use of the information in creating genetic tests. The algorithms also encourage additional research and work in proving the associations between a genetic variant and phenotypic result, which leads to higher data quality and curation scores and thereby increased revenue for genetic testing. By providing submitters of information payment based on the submissions, the system and methods described herein encourage researchers and clinicians to submit their genetic research in order to obtain payment for any genetic test that is eventually created based on that information.

The algorithms also encourage making submitted data publicly available by only awarding points, and therefore payment, for visible submissions, or by reducing the points awarded to invisible submissions.

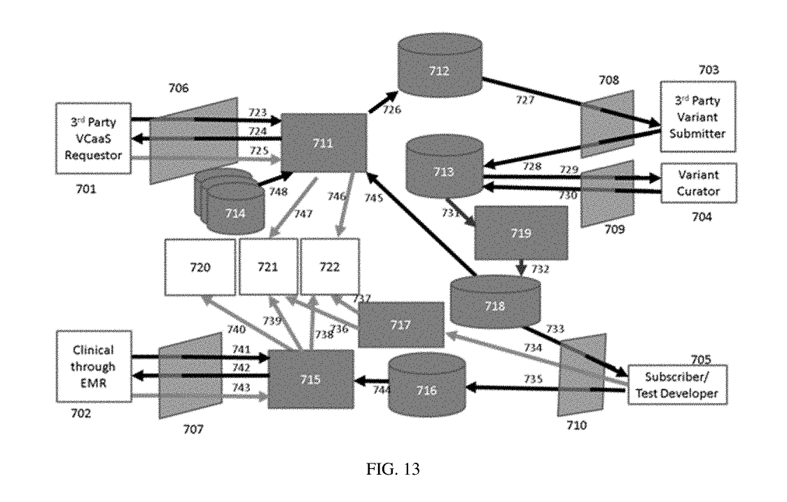

FIG. 13 illustrates an overview of a system for curation and analysis of genetic variants. A requestor 701 submits a curation request through a curation request application portal 706 to a variant curation interpreter 711, as illustrated by arrow 723. As part of the request, the requestor 701 provides access to a sample's genetic information and phenotype. The variant curation interpreter 711 obtains known non-reference genetic variants with the known associated classification from public curated and scored databases 714, as shown by arrow 748, as well as proprietary curated and scored databases 718, as shown by arrow 745. The variant curation interpreter 711 assesses the sample's genetic information based on the public and proprietary classification of non-reference genetic variants. The variant curation interpreter 711 returns a curation report back to the requestor 701 through the curation request application portal 706, as shown by arrow 724. The curation report can summarize the classification of the non-reference genetic variants. The requestor 701 can pay into the system for the curation report, as shown by arrow 725. The system can also account for payment of royalties to variant owners 722 for use of the proprietary information in obtaining the curation report, as shown by arrow 746. The system can also account for a payment to data sample owners 721 for data samples used as supporting evidence for the variants used by the variant curation interpreter 711 in the proprietary curated and scored databases 718, as shown by arrow 747. Non-reference genetic variants from the requestor's sample which do not appear in the public or proprietary variant databases may be logged to a data sample database 712 as shown by arrow 726. The non-reference genetic variants and sample phenotypes can be stored for future use.

Optionally, the system can allow a third-party submitter 703 to review the non-reference genetic variants from the data sample database 712 through a submission application 708 as indicated by arrow 727. The third party submitter 703 can also submit a variant for review and curation into a genetic information database 713 as indicated by arrow 728. The third party submitter 703 may submit their own supporting data, supporting data available from the data sample database 712, or a combination of their own supporting data and data available from the data sample database 712.

One or more curators 704 can retrieve a new variant submission from the genetic information database 713 through curation application portal 709 as indicated by arrow 729, and the curators 704 can evaluate the strength of the variant submission per curation guidelines. Based on the evidence, the curators 704 return a curation score to the genetic information database 713, as indicated by arrow 730. Multiple instances of the same curated variant can be reviewed by a scoring application 719 as indicated by arrow 731 and scored to determine the official curated variant classification. A single instance of the curated variant is scored and stored in the he proprietary curated and scored databases 718 as indicated by arrow 732. One of skill in the art will understand that the proprietary curated and scored databases 718 can be the same database as the genetic information database 713, or can be a separate database as illustrated in FIG. 13.

Third parties, such as test developers or subscribers 705 interested in the content of the proprietary curated and scored databases 718 can subscribe to access the information through a test developer or subscription application portal 710, as indicated by arrow 733. The test developers or subscribers 705 can be required to pay a periodic subscription fee to access the information in the proprietary curated and scored databases 718, as indicated by arrow 734. A payment application 717 can account for a fractional royalty payment to the data sample owners 721 as indicated by arrow 736, and to variant owners 722 as indicated by arrow 737, for the content of the proprietary curated and scored databases 718 based on the quality of the content as determined by the curation score. As described, the test developers or subscribers 705 can also create and submit a genetic test to a proprietary test database 716 as indicated by arrow 735. The test can be submitted through the subscription application portal 710 or through a separate test submission application. The genetic test is made available for clinical use.

To conduct a genetic test, a clinician 702 submits a request for a genetic test available in the proprietary test database 716 through a test request application portal 707, as indicated by arrow 741. A genetic data interpretations server 715 retrieves instructions for the genetic test from the proprietary test database 716 as indicated by arrow 744. The genetic data interpretations server 715 executes the genetic test based on the test instructions. The genetic data interpretations server 715 then returns the genetic test results to the clinician 702 as indicated by arrow 742. A payer party pays for the genetic test as indicated by arrow 743. A payment application can determine the payments due to the data sample owners 721 as indicated by arrow 739, to the variant owners 722 as indicated by arrow 738, and to the test developers 720 as indicated by arrow 740.

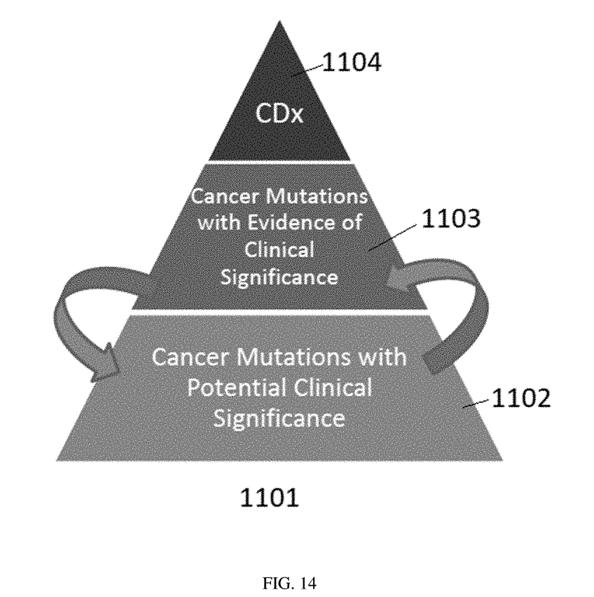

FIG. 14 illustrates a use case showing the potential benefits of the described systems. The Federal Food and Drug Administration (FDA) classifies tests based on the potential adverse effects if results are incorrect according to pyramid 1101. At the top of the pyramid 1104 are companion diagnostics, requiring the highest amount of evidence for approval. The middle section of the pyramid 1103 are moderate risk tests and require evidence of clinical significance for the tests based on professional guidelines, such as the ACMG guidelines. The bottom level of the pyramid 1102 are low risk tests for cancer mutations, which require the lowest amount of evidence, such as literature or mechanistic rationale for inclusion in a panel. The FDA allows flexibility to parties to report mutations based on current knowledge. That is, as more evidence is gathered, certain mutations may move from the lowest level of the pyramid 1102 to the middle level of the pyramid 1103, or vice versa, without additional reporting to the FDA. Test developers can use the described systems to gather additional evidence of clinical significance of genetic variants and can use the current knowledge of the clinical significance in FDA reporting as genetic variants are curated. The ability to obtain royalties for work put in by the independent curators incentivizes curation, resulting in more accurate genetic tests.

The software implementing the above processes can be coded in any language known in the art, including, but not limited to, ASP, APS.NET, Java, JavaScript, C, C++, C#, C#.NET, Objective C, F#, F#.NET, Basic, Visual Basic, VB.NET, Go, Python, Perl, Hack, PHP, Erlang, XHP, Scala, Ruby, J2EE, SQL, CGI, HTTP, or XML.

It will be apparent to one skilled in the art that various combinations and/or modifications and variations can be made in the system depending upon the specific needs for operation. Moreover, features illustrated or described as being part of one embodiment may be used on another embodiment to yield a still further embodiment.

We Claim:

1. A system, comprising:
a genetic information database; wherein the genetic information database contains genetic variants and estimated effects of the genetic variants;
a curation application, wherein the curation application is connected to the genetic information database, and wherein the curation application is accessible by one or more curators and allows access to the genetic information database;
wherein the curation application allows the one or more curators to curate information in the genetic information database and allows the one or more curators to provide a curation score for the information in the genetic information database;
a submission application; wherein the submission application is connected to the genetic information database; and wherein the submission application is accessible by one or more submitters;
wherein the submission application allows the one or more submitters to submit the genetic variants and estimated effects of the geneticvariants to the genetic information database; and
a payment application, wherein the payment application is programmed to account for a payment to the one or more curators and the one or more submitters.

2. The system of claim 1, wherein the payment application either accounts for the payment to the one or more curators each time a variant curated by a curator is viewed, each time a curator curates a variant, or a combination thereof.

3. The system of claim 1, wherein the payment application accounts for the payment to the one or more submitters either each time a variant submitted by a submitter is used in a genetic test, each time a variant submitted by a submitter is viewed, or a combination thereof.

4. The system of claim 2, wherein the system is programmed to use a block chain to account for the payment to the one or more curators.

5. The system of claim 4, wherein the system is programmed to update the blockchain each time a variant is curated by a different curator.

6. The system of claim 4, wherein the system is programmed to account for the payment to the one or more curators by a fiat currency, a utility token, or a security token.

7. The system of claim 3, wherein a payment to the one or more submitters is based on a variant score for a submission.

8. The system of claim 7, wherein the system is programmed to use a block chain to account for the payment to the one or more submitters.

9. The system of claim 8, wherein the system is programmed to account for the payment to the one or more submitters a fiat currency, a utility token, or a security token.

10. The system of claim 1, further comprising a test submission application; wherein the test submission application is connected to a genetic data interpretations server; and wherein the test submission application allows one or more test developers to submit a genetic test; wherein the genetic test comprises instructions for determining a presence, absence, or likelihood of a genetic condition; and wherein the genetic test is based on one or more variants in the genetic information database.

11. The system of claim 10, wherein the genetic data interpretations server is collocated with a genetic data storage server and a remote client; the genetic data storage server containing a genome or portion of a genome for one or more patients; the remote client programmed to conduct a genetic test based on information in the genetic data interpretations server.

12. The system of claim 10, wherein the payment application is programmed to account for a payment to the one or more test developers each time a genetic test developed by the one or more test developers is conducted.

13. The system of claim 10, wherein the payment application is programmed to account for a payment to the one more submitters each time a genetic test is conducted using a variant submitted by the one or more submitters, and to account for a payment to the one more curators each time a genetic test is conducted using a variant curated by the one or more curators.

14. The system of claim 13, wherein the system is programmed to use a block chain to account for the payment to the one or more submitters.

15. The system of claim 14, wherein the system is programmed to account for the payment to the one or more submitters by a fiat currency, a utility token, or a security token.

16. The system of claim 13, wherein the payment to the one or more submitters is based on a price for the genetic test and a variant score for each variant submitted by the one or more submitters used in the genetic test.

17. The system of claim 10, wherein the payment application is programmed to account for a payment from a payer party to the one or more test developers each time a genetic test developed by the one or more test developers is conducted, to the one or more curators each time a genetic test using a variant curated by the one or more curators is conducted, and to the one or more submitters each time a genetic test using a variant submitted by the one or more submitters is conducted.

18. The system of claim 1, wherein the payment application accounts for a payment from a subscriber for viewing one or more variants in the genetic information database.

19. The system of claim 19, wherein the payment application is programmed to distribute the payment from the subscriber to the one or more suhrriitters and one or more curators according to an algorithm.

20. The system of claim 1, further comprising a curation database, the curation database containing curation information submitted by the one or more curators.
